(19) United States
(12) Patent Application Publication
(10) Pub. No.: US 2009/0024060 A1
(43) Pub. Date: Jan. 22, 2009

(54) SAMPLE COLLECTOR

(76) Inventors: William A. Darrigrand, Nazareth, PA (US); Kristen A. Blum, Whitehall, PA (US); Craig W. Breckenridge, New Tripoli, PA (US); Gunnar Finberg, Quakertown, PA (US); Philip M. Formica, Center Valley, PA (US); Dean Francis Fritch, East Greenville, PA (US); Keith W. Kardos, Bethlehem, PA (US); Stephen Lee, Doylestown, PA (US); Vijaya K. Mokkapati, Macungie, PA (US); John William Scott, Perkasie, PA (US); Daniel G. Shenberger, Coopersburg, PA (US)

Correspondence Address: NIXON PEABODY, LLP, 401 9TH STREET, NW, SUITE 900, WASHINGTON, DC, 20004-2128, US

(21) Appl. No.: 12/104,311

(22) Filed: Apr. 16, 2008

Related U.S. Application Data
(60) Provisional application No. 60/907,757, filed on Apr. 16, 2007.

(51) Int. Cl. A61B 5/00

(52) U.S. Cl. 600/584; 600/573

(57) ABSTRACT

A collector includes a collecting element that receives a sample of bodily fluid. The collecting element may be an absorbent pad that has been treated with a surfactant to optimize recovery of analytes from the sample and/or their absorbance onto the absorbent material. An extractor is operably connected to a container and receives the collector to provide fluid communication between the collector and the container. The collector, when received by the extractor, is operable to release a volume of the sample into the container.

### CROSS REFERENCE TO RELATED APPLICATIONS

This application claims priority to U.S. Provisional Application No. 60/907,757 filed Apr. 16, 2007, the contents of which are incorporated entirely herein by reference.

### BACKGROUND OF THE INVENTION

1. Field of the Invention

The invention relates generally to a system for collecting samples of bodily fluids, and particularly to a system that employs a collector for receiving a sample and an extractor for releasing the sample into a container for preservation, transport, and testing. More particularly, the invention also relates to an oral-fluid collecting element, such as an absorbent pad, having improved recovery for drugs-of-abuse metabolites present in oral fluid.

2. Description of Related Art

Samples of bodily fluids, such as blood, urine and saliva, may be collected in a number of ways in order to test for the presence of analytes. One type of sample collector typically includes an absorbent pad for absorbing the target fluid and a holder for holding the sample as the sample is being collected. Once the sample is absorbed by the absorbent pad, the entire pad is transferred to a vial. The vial is then delivered for testing. Disadvantageously, these systems still require additional manipulation, such as centrifugation of the sample in the vial, before the sample can be tested. Other types of sample collectors may release, or express, the sample from the absorbent pad into the vial, rather than placing the entire pad in the vial. Alternatively, the sample may be introduced directly into a testing device, such as a lateral test flow device, rather than storing the sample in a vial for subsequent testing. In particular, with typical devices, a precise quantity of oral fluid is not delivered. A metered quantity of oral fluid is critical to ensure that the quantity is sufficient for testing purposes and to allow determination of actual oral fluid concentrations when the oral fluid is combined with a preservative solution.

As discussed, samples of bodily fluids may include saliva. Humans produce up to 1.5 liters of saliva each day. The use of saliva or “oral fluid” samples is well established for substance of abuse or drug testing and disease testing. Collecting oral fluid specimens is generally considered to be less invasive and less embarrassing, and less stigmatizing than the collection of other bodily fluids, such as blood, serum, urine, etc. The term “oral fluid” is generally considered a better descriptor than “saliva” for the fluid collected in oral specimens. Oral fluids are produced from multiple glands in the mouth. Oral fluid is made up of both saliva and mucosal excretions. Oral fluids contain glandular and cellular debris present in the oral cavity as well as components of blood which include antibodies and drug metabolites.

Previous oral fluid collection devices have been designed to stimulate oral fluid production or increase the absorbency of the collection pad. For example, pads treated with a hypertonic salt solution are described in U.S. Pat. No. 5,103,836. While stimulation of oral fluid production and increasing pad absorbency improve sampling of oral fluids, the collected sample must also be successfully recovered from the collection pad for testing. Some analytes, such as tetrahydrocannabinol (THC) from marijuana, tend to bind to the collection pad. This can result in inaccurate measurement of the amount of analyte present in the oral fluid.

### SUMMARY OF THE INVENTION

Embodiments according to aspects of the present invention provide a system that improves the process of releasing, or expressing, a sample of bodily fluid from a collection device. In particular, embodiments enable users to manipulate the collection device to release an appropriate volume of sample fluid, which can be tested, for example. Moreover, embodiments facilitate sample processing at the testing site of the sample fluid which is stored and delivered in a vial. For instance, centrifugation may be eliminated as a necessary processing step.

Accordingly, an embodiment has a collector having a sample collecting element. The sample collecting element receives a sample of bodily fluid when the collector is in a first configuration and releases the sample when the collector transitions from the first configuration to a second configuration. In addition, the embodiment also has a container adapted to receive and store the sample released from the collector. Moreover, the embodiment has an extractor operably connected to the container, where the extractor receives the collector and has a passage providing fluid communication between the collector and the container. The collector, when received by the extractor, is operable to transition from the first configuration to the second configuration and release a predetermined volume of the sample. The container may have a diluent into which the sample is received and stored. For example, the diluent may be a preservative that maintains the integrity of the sample during storage and transport.

In a particular embodiment, the collecting element is an absorbent pad, such as a polyolefin fiber pad pretreated with a buffered salt, that is applied to a source of bodily fluid to collect a sample. In addition, the collector may include an indicator that informs the user when a sufficient sample volume has been collected or absorbed. The sample is then released from the collecting element by compressing the collecting element with a plunger. The compression by the plunger may be achieved by various techniques described below. In this embodiment, the first configuration corresponds with the collector leaving the absorbent material uncompressed, while the second configuration corresponds with the collector compressing the collecting element to release the sample from the collecting element. The sample may be released from the collector partially or in a controlled manner by operating the collector to occupy states between the first and second configurations. In particular, the collecting element may have varying states of compression as the collector is transitioned from the first to second configurations.

A further embodiment employs an indicator that indicates the volume of sample released from the collector into the container. In particular, the indicator may be a window showing a level of fluid in the container. Therefore, the user may control the amount of sample fluid being released into the container by observing the indicator.

Yet a further embodiment employs an overflow chamber positioned in at least one of the collector and the extractor. The overflow chamber has an overflow opening to receive an excess volume of the sample when the collector is operated to transition from the first configuration to the second configuration. In this way, the volume of sample fluid introduced into the container is controlled.

As described previously, bodily fluid samples collected by embodiments may include saliva, or oral fluid. Accordingly, a further aspect of the present invention relates to a method of collecting an oral fluid specimen from an oral cavity for testing. While the method is preferably designed to obtain oral fluid samples to test for drugs of abuse in human subjects, the method may be used to obtain oral fluid sample from humans for other purposes or to obtain oral fluid samples from animals. Moreover, collectors in embodiments of the present invention may employ a collecting element, such as an absorbent pad, that is treated to optimize recovery of analytes from the sample. Therefore, according to an embodiment, a compressible, detergent-treated collecting element is inserted into the oral cavity of the subject's mouth to collect an oral fluid sample. The fluid sample may then be released, or expressed, from the collecting element into a container in a manner employing the systems and devices described herein. However, such treatment may also be applied more broadly to any system or device for collecting samples of fluid.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1A illustrates an example embodiment according to aspects of the present invention.

FIG. 1B illustrates an exploded view of the collector of the example embodiment shown in FIG. 1A.

FIG. 1C illustrates a cross-sectional view of the extractor and the container of the example embodiment shown in FIG. 1A.

FIG. 1D illustrates a cross-sectional view of the container and the cap of the example embodiment shown in FIG. 1A.

FIG. 1E illustrates a cross-sectional view of the collector, the extractor, and the container of the example embodiment shown in FIG. 1A.

FIG. 2A illustrates a cross-sectional view of another example embodiment according to aspects of the present invention, with an uncompressed collecting element.

FIG. 2B illustrates a cross-sectional view of the example embodiment of FIG. 2A, with a compressed collecting element.

FIG. 2C illustrates a cross-sectional view of the container and the cap of the example embodiment of FIG. 2A.

FIG. 3A illustrates a cross-sectional view of yet another example embodiment according to aspects of the present invention, with an uncompressed collecting element.

FIG. 3B illustrates a cross-sectional view of the example embodiment of FIG. 3A, with a compressed collecting element.

FIG. 3C illustrates a cross-sectional view of the container and the cap of the example embodiment of FIG. 3A.

FIG. 3D illustrates a cross-sectional view of an alternative collecting element, without an overflow chamber, useable in the example embodiment of FIG. 3A.

FIG. 4A illustrates a cross-sectional view of another example embodiment according to aspects of the present invention, with an uncompressed collecting element.

FIG. 4B illustrates a cross-sectional view of the example embodiment of FIG. 4A, with a compressed collecting element.

FIG. 5A illustrates a cross-sectional view of a further example embodiment according to aspects of the present invention, with an uncompressed collecting element.

FIG. 5B illustrates a cross-sectional view of the example embodiment of FIG. 5A, with a compressed collecting element.

FIG. 5C illustrates a cross-sectional view of the example embodiment of FIG. 5A, with an overflow chamber.

FIG. 6A illustrates a cross-sectional view of yet another example embodiment according to aspects of the present invention, with an uncompressed collecting element.

FIG. 6B illustrates a cross-sectional view of the example embodiment of FIG. 6A, with a compressed collecting element.

FIG. 6C illustrates a transparent view of open overflow chambers in the example embodiment of FIG. 6A.

FIG. 6D illustrates a transparent view of closed overflow chambers in the example embodiment of FIG. 6A.

FIG. 6E illustrates a transparent view of the example embodiment of FIG. 6A, with a broken seal to the container.

FIG. 7A illustrates an exploded view of another example embodiment according to aspects of the present invention.

FIG. 7B illustrates a cross-sectional view of the collector for the example embodiment of FIG. 7A.

FIG. 7C illustrates a cross-sectional view of the container for the example embodiment of FIG. 7A.

FIG. 7D illustrates the collector in combination with the container for the example embodiment of FIG. 7A.

### DETAILED DESCRIPTION

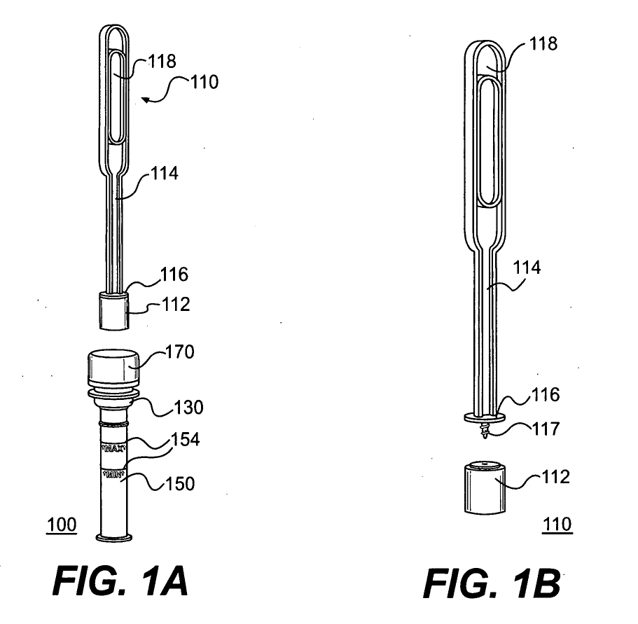

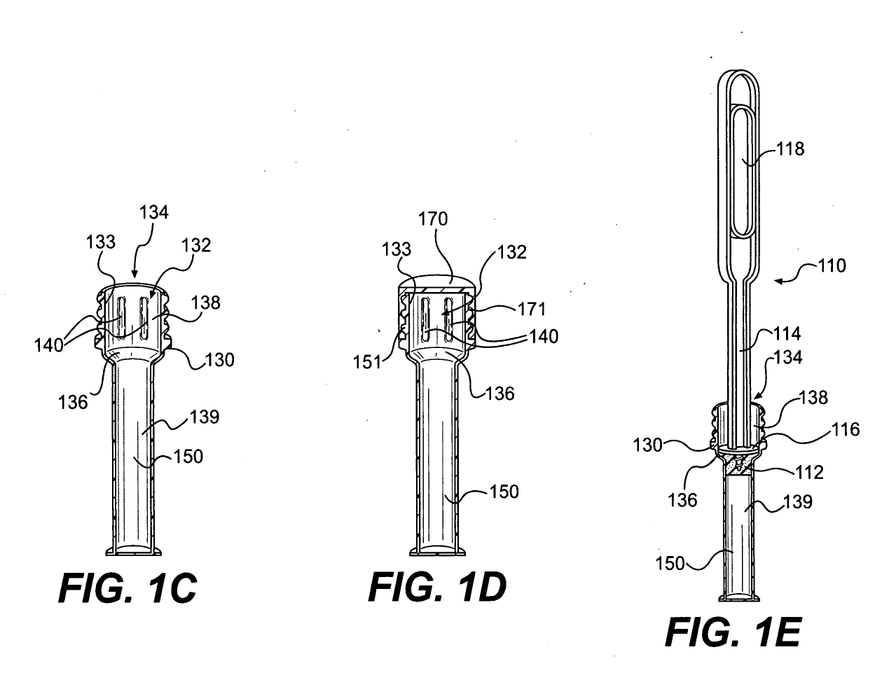

FIG. 1A illustrates an exemplary embodiment of the present invention. In particular, a system 100 for collecting samples of bodily fluid employs a collector 110, an extractor 130, the storage container 150, and a cap 170.

The collector 110 employs an element 112 adapted to receive a sample from a source of bodily fluid. Samples of bodily fluids include, but are not limited to, saliva, urine, or blood. The collecting element 112 may be a pad, sponge, or the like, formed from an absorbent material. The absorbent material may include natural occurring absorbent materials such as cotton or cellulose materials as well as synthetic fibers such as, but not limited to, polyesters. As such, when the collecting element 112 is applied to, or placed into contact with, a source of fluid, it absorbs some of the fluid from the source. In addition, the collecting element 112 may be treated with a surfactant (or detergent) to optimize recovery of analytes from the sample and/or their absorbance onto the absorbent material. The classes of surfactants that can be used in accordance with the invention include, nonionic, cationic, anionic or zwitterionic surfactants, such as but not limited to Brij 35, Tween-20, Tween-60, Tween-80, PEG-80, PEG-400, or Triton X100. Food grade surfactants are preferred. Nonionic surfactants, such as PEG surfactants, particularly PEG esters, are particularly preferred. The surfactants should preferably have no taste or very little tasted when used to collect oral fluid samples. To treat the absorbent material, the collecting element 112 is allowed to absorb a solution of the surfactant until it is saturated and then dried. The amount of surfactant used to treat the collecting element 112 may be varied by changing the concentration of the surfactant in the solution. Where the surfactant has an unpleasant taste, a flavorant or sweetener, as is known in the art, may be added to mask the unpleasant taste. Additionally buffering agents and other agents used in the art to treat bodily fluid samples, particularly oral fluid samples may be dried onto the collecting element 112 with the surfactant.

The collecting element 112 is initially sized so that a sufficient volume of the sample fluid may be absorbed from the fluid source. The presence of the sample fluid in the collecting element 112 may cause the collecting element 112 to expand in size. In addition, the collecting element 112 generally holds the sample until the collecting element 112 is manipulated to release, or express, the sample. For example, the sample held by the collecting element 112 may be released from the absorbent material by compressing the collecting element 112, thus reducing the volume of the collecting element 112 and its ability to hold the sample.

The embodiments described herein generally receive a sample of bodily fluid when a collector is in a first configuration and release the sample when the collector transitions from the first configuration to a second configuration. In some embodiments, the sample may be released from the collector partially or in a controlled manner by operating the collector to occupy states between the first and second configurations. For example, a collecting element may have varying states of compression as the collector is transitioned from the first to second configurations.

Referring again to FIG. 1A, the collecting element 112 may be substantially cylindrical in shape, but the collecting element 112 is not limited to this particular shape. For instance, the shape of the collecting element 112 may have a substantially oval profile to facilitate application of the collecting element 112 between the cheek and gums, if saliva samples are to be collected. In general, the shape of collecting element 112 corresponds to the shapes of the extractor 130 and container 150 so that the collector 110 may be employed with the extractor 130 and container 150, as described further below. However, it is understood that although the embodiments described herein may employ cylindrical or other shapes, it is understood that the shapes employed by embodiments of the present invention are not limited to a cylinder or a particular shape.

As further illustrated in FIG. 1A, the collecting element 112 is attached to an end 116 of a plunger 114. The various techniques that may be employed to attach the collecting element 112 to the plunger end 116 include, but are not limited to, the use of adhesives, chemical bonding, fasteners, mechanical joining, or the like, or any combination thereof. For example, as shown by the exploded view of the collector 110 in FIG. 1B, a protrusion, or barb, 117 extending from the plunger end 116 may be employed to pierce the collecting element 112 and hold the collecting element 112 against the plunger end 116 in frictional engagement. Alternatively, the collecting element 112 may also be attached to the plunger end 116 with an adhesive, while the protrusion 117 also ensures that the collecting element 112 is stably positioned and remains aligned with the plunger 114 to facilitate use of the collector 110 with the extractor 130 and container 150.

As shown in FIG. 1B, the end 116 of the plunger 114 may be substantially disc-shaped to correspond with the substantially cylindrical shape of the collecting element 112. However, the plunger end 116 is not limited to this particular shape. In general, the plunger end 116 is shaped to enable the plunger 114 to be operated to apply pressure to an entire side, e.g. the top side, of the collecting element 112, as described further below.

The plunger 114 also has a longitudinal handle 118 which extends from the plunger end 116. The plunger handle 118 enables a user to operate, or manipulate, the collector 110 to cause the sample to be released, or expressed, from the collecting element 112, as described further below. In addition, the plunger handle 118 allows a user to apply the collector 110 to the source of bodily fluid while minimizing any contact between the user and the source. Moreover, contact between the user and the collecting element 112 is minimized, helping to prevent contamination of the sample.

Referring again to FIG. 1A, the collection system 100 employs a substantially cylindrical container, or vial, 150 to receive and store the sample collected by the collecting element 112. The container 150 may contain a diluent, such as a surfactant containing solution (such as Tween 20) with a preservative (such as Chlorhexadine or Proclin 5000), with which the sample may be stored. Advantageously, the diluent provides stabilization and dilution of the sample for processing at the testing site. Additionally the diluent provides a pretreatment of the sample to minimize matrix effects. For instance, in an exemplary embodiment collecting saliva samples, approximately 2 ml of buffer may be stored in the container 150.

The extractor 130 is employed to operate the collector 110 and release, or express, the sample from the collector 110 into the container 150. In the particular embodiment illustrated in FIG. 1A, the extractor 130 may be integrally formed with the container 150, for example, from a molded plastic. However, in alternative embodiments, the extractor 130 and the container 150 may be separately formed but subsequently joined together.

As shown in the cross-sectional view of FIG. 1C, the extractor 130 is positioned at an upper portion of the container 150. The extractor 130 has an extractor cavity 132 defined by a wall 133. The extractor cavity 132 has an upper opening 134, through which the collector 110 is received. FIG. 1C shows that the wall 133 of the cavity 132 has an annular section, or step, 136 that causes the wall 133 to turn transversely inward. As a result, the diameter, or width, of the cavity 132 narrows at a distance from the opening 134, so that the extractor has two substantially cylindrical sections 138 and 139 having different diameters. Thus, the collecting element 112, extractor 130, and the container 150 are defined by substantially cylindrical shapes. The first cylindrical section 138 has a diameter that is larger than the diameter of the collecting element 112, and the second cylindrical section 139 has a diameter that is smaller than the diameter of the collecting element 112. In addition, the diameter of the second cylindrical section 139 is substantially equal to the diameter of the container 150.

Additionally, the wall 133, above the annular section 136, may also have grooves 138 that facilitate the introduction of the collector 110 into the extractor 130. The grooves 138 guide the positioning of the collector 110 in the extractor 130, and also allow air to escape upwards along the wall 133 to reduce the amount of pressure acting on the collector 110 as it is moved into the extractor 130.

As shown in the cross-sectional view of FIG. 1D, the cap 170 may engage the container 150 with corresponding screw threads 151, 171 on the container 150 and the cap 170, respectively. However, cap 170 may engage the container 150 according to other techniques including, but not limited to, a snap fit, tight frictional engagement, or the like.

In operation, the user holds the collector 110 by the handle 118 and maneuvers the collecting element 112 into contact with a source of bodily fluid. For example, the collecting element 112 may be applied or swabbed inside the mouth, in contact with the gums, to receive a sample of saliva. In particular, once the absorbent material of the collecting element 112 comes into contact with the fluid source, some of the fluid is drawn, or absorbed, into the collecting element 112. In order to collect a sufficient volume of the sample fluid, the collecting element 112 may have to remain in contact with the fluid source for a specified amount of time.

After the user applies the collecting element 110 to absorb the sample, the user, while holding the collector 110 by the handle 118, introduces the collecting element 112 into the extractor 130 in order to release the sample into the container 150. In particular, the collecting element 112 is passed through the opening 134 into the first cylindrical section 138 of the extractor 130. The collecting element 112 is directed further into the extractor 130, where the wall 133 may guide movement of the collector 110. Because the diameter of the first cylindrical section 138 is larger than the diameter of the collecting element 112, the collecting element 112 remains substantially uncompressed while in the cylindrical section 138. Moreover, because the plunger end 116 has a diameter substantially equal to the diameter of the collecting element 112, the plunger end 116 minimizes any transverse compression of the collecting element 112 from contact with the wall 133 in the cylindrical section 138.

With further introduction of the collecting element 112 into the first cylindrical section 138, the collecting element 112 moves into abutment with the annular section 136 of the extractor 130. Because the inner diameter of the annular section 136 is smaller than the diameter of the collecting element 112, the annular section 136 makes contact with an outer portion of the bottom surface of the collecting element 112, resulting in compression of the collecting element 112 in the longitudinal direction and a release of the sample fluid from the collecting element 112. A slight transverse movement, or pressure, against the wall 133 may be employed to allow fluid to be released into the container 150 while allowing for air to escape. Some portion of the collecting element 112 may enter the cylindrical section 139, but the collecting element 112 generally remains in abutment with the annular section 136 to cause the longitudinal compression.

As shown in the cross-sectional view of FIG. 1E, the collecting element 112 is compressed in abutment with the annular section 136. At some point, the collector 110 cannot proceed any further into the extractor 130, such as may be limited by the optional protrusion 117. The collecting element 112 has been substantially compressed to a maximum, or near maximum, extent. As there is no further compression, no more sample fluid is released from the collecting element 112.

As shown in FIG. 1A, the container 150 has an indicator 154 that shows the user how much sample fluid has been released into the container 150, so that the user may operate the plunger 114 accordingly to obtain a more optimal volume of sample fluid. In particular, the indicator 154 may be a window that shows the minimum and maximum amount of sample fluid appropriate for testing. An appropriate amount of fluid has been released into the container 150 if the fluid level can be seen in the window. For instance, in an exemplary embodiment collecting saliva samples, an appropriate amount of saliva may have a range of approximately 1 to 2 ml. Additionally, a frosting along the side of the container 150 may have a vertical clear section to allow continual monitoring while the sample is being received into the container 150.

Taking into account the expected amount of absorption and expansion by the collecting element 112 and the amount of compression caused by the extractor 130, the amount of sample fluid released from the collecting element can be roughly estimated. Thus, the collecting element 112, the annular section 136, and the diameter of the second cylindrical section 139 may be configured so that the collection system 110 yields an acceptable amount of sample fluid.

When the collector 110 is operated to release the sample from the collecting element 112, the sample enters the container 150, which is in fluid communication with the collector 110 via the extractor 130. Once the release of the sample is complete, the collector 110 may be removed from the extractor 130. The cap 170 may then be employed to seal the container 150 and protect the integrity of the sample. Once the container 150 is sealed, the sample may be stored in the container 150 and delivered for testing.

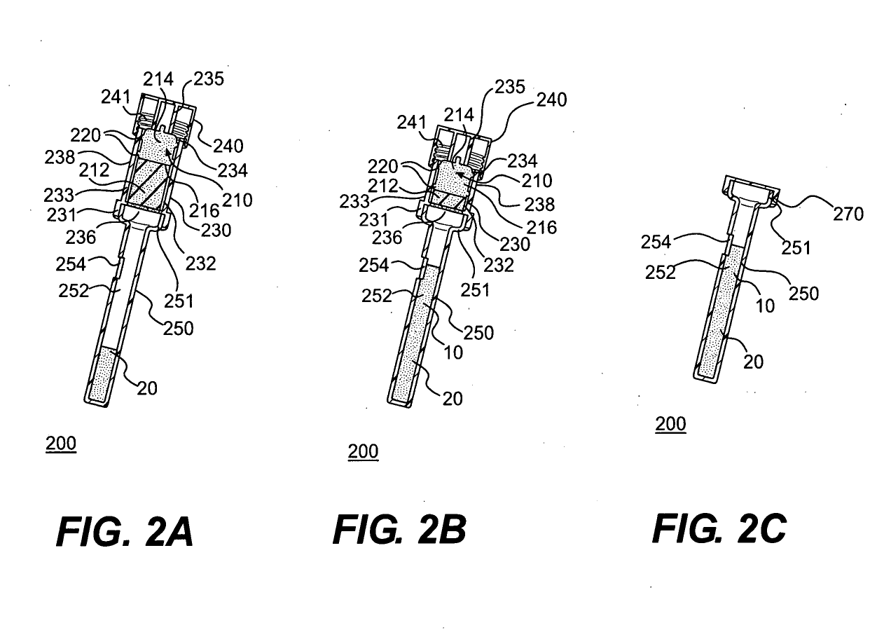

FIG. 2A illustrates another embodiment of the present invention. In particular, the collection system 200 includes a collector 210, an extractor 230, and container 250.

The collector 210 has an element 212 adapted to receive a sample from a source of bodily fluid. Similar to the collecting element 112 of the collector system 100 described previously, the collecting element 212 may be a treated or untreated sponge, pad, or the like, formed from an absorbent material. Like the collecting element 112, the collecting element 212 is attached to an end 216 of a plunger 214. As shown in FIG. 2A, the plunger end 216 may be substantially dome-shaped with a substantially circular bottom surface that corresponds with the top of the substantially cylindrical shape of the collecting element 212.

The plunger 214 has a longitudinal handle (not shown) which extends from the plunger end 216. Similar to the handle 118 described previously, the handle of plunger 214 enables a user to operate, or manipulate, the collector 210. However, unlike the handle 118, the handle of plunger 214 is detachable from the plunger end 216. FIG. 2A shows collector 210 including the collecting element 212 attached to the plunder end 216 without the plunger handle.

Referring again to FIG. 2A, the collection system 200 employs a substantially cylindrical container 250 to receive and store the sample from the collecting element 212 of the collector 210. The container 250 contains a diluent 20, such as a surfactant containing solution (for example, Tween 20) with a preservative (for example, Chlorhexadine or Proclin 5000), with which the sample may be stored.

The extractor 230 is employed to operate the collector 210 and release, the sample from the collector 210 into the container 250. In the particular embodiment illustrated in FIG. 2A, the extractor 230 includes a receptacle 238 and an extractor cap 240. The receptacle 238 is detachably connected to the container 250. A bottom mating section 231 of the receptacle 238 fits over an upper mating section 251 of the container 250 with a substantially watertight seal. The bottom mating section 231 and the upper mating section 251 may have corresponding substantially cylindrical shapes. The fit may be achieved by various techniques including, but not limited to, a snap fit, tight frictional engagement, screw-threads, or the like.

The receptacle 238 has an extractor cavity 232 defined by a wall 233. The extractor cavity 232 also has an upper opening 234, through which the collector 210 is received. As shown in FIG. 2A, the extractor cavity 232 is substantially cylindrical and is sized to receive the collecting element 212 attached to the plunger end 216 which has been detached from the plunger handle. The plunger end 216 also has annular seals 220 which guide the plunger end 216 along the wall 233 and minimize the amount of fluid that escapes past the plunger end 216.

The extractor cap 240 fits over the upper opening 234 of the receptacle 238. In particular, the extractor cap 240 engages the receptacle 238 via screw-threads 241. A plunger contact piece 235 on the interior of the cap 240 extends from the inside surface of the extractor cap 240. As the extractor cap 240 is rotated, or screwed, onto the receptacle 238, the extractor cap 240 moves into downward engagement with the receptacle 238. Correspondingly, the plunger contact piece 235 moves downward with the extractor cap 240. As described further below, the downward movement of the plunger contact piece 235 is employed to release the sample from the collecting element 212 in the receptacle 238. It is understood that technique for engagement of the extractor cap 240 with the receptacle 238 is not limited to screw-threads 241, and any mechanism that allows the extractor cap 240 to be operably connected to, and guided downwardly with respect to, the receptacle 238 may be employed.

The receptacle 238 has a screen 236 which separates the interior cavity 232 of the extractor 230 and the interior cavity 252 of the container 250. The screen 236 may be injection molded, and it may be a separate piece which is added as part of the device assembly. The screen 236 provides fluid communication between the interior cavities 232 and 252. When the collecting element 212 is introduced into the receptacle 238, the collecting element 212 is positioned adjacent to the screen 236.

In operation, the user applies the collector 210 to collect a sample of bodily fluid in a manner similar to the application of the collector 110 described previously. After the sample is collected with the collecting element 212, the user, while holding the collector 210 by the handle, positions the collecting element 212 and the plunger end 216 in the receptacle 238. The user then detaches the handle from the plunger end 216. In one possible embodiment, the handle may be detached by breaking, or snapping, it off from the rest of the collector 210. Alternatively, the handle may be reversed out of a mechanical interlock with the plunger end 216. The contact between the seals 220 and the wall 233 of the receptacle 238 may help hold the plunger end 216 in place while the handle is detached.

Once the handle has been detached, the user positions the extractor cap 240 over the receptacle opening 234 and rotates the extractor cap 240 so that the extractor cap 240 engages the receptacle 238 via screw-threads 241. The screw-threads 241 force the extractor cap 240 downward into further engagement with the receptacle 238. Correspondingly, the plunger contact piece 235 moves downward. Initially, the plunger contact piece 235 makes contact with the top of the dome-shaped plunger end 216. Continued rotation and downward movement of the extractor cap 240 and the contact piece 235 then causes the plunger contact piece 235 to apply pressure to the collecting element 212 and force the collecting element 212 into abutment with the screen 236. As shown in FIG. 2B, further downward movement of the extractor cap 240 and the contact piece 235 causes longitudinal compression of the collecting element 212 between the plunger end 216 and the screen 236. With this compression, the size of the collecting element 212 and the volume of fluid the collecting element 212 can hold are reduced. As a result, some of the sample fluid in the collecting element 212 is released. In this way, the threaded extraction cap 240 controls the rate of release, or expression. During operation of extractor cap 240, some escape of air from the system 200 is required in order to permit the air in the container 250 to escape and be displaced by sample fluid from the collecting element 212.

When the extractor 230 is operated to release the sample from the collecting element 212, the sample passes through the screen 236 into the container 250. The seals 220 help to ensure that the sample fluid released from the collecting element 212 does not escape along the wall 233 past the plunger end 216. Advantageously, the screen 236 reduces aeration of the sample as it enters the container 250. The extractor cap 240 is operated to compress the collecting element 212 until a sufficient volume of the sample is released. An indicator 254, such as a mark or window, on the container 250 may be employed to alert the user when enough of the sample has been released. FIG. 2B illustrates the compression of collecting element 212 to release the sample 10 until the sample 10 is approximately level with the indicator 254. The diameter of the container 250 may be small in order to make changes in the volume of sample 10 in the container 250 more evident.

Once the appropriate sample volume 10 is received into the container 250, the extractor 230 may be removed from the container 230. As FIG. 2C shows, a container cap 270 may then be employed to seal the container 250 and protect the integrity of the sample. Once the container 250 is sealed, the sample may be stored in the container 250 and delivered for testing. In an alternative embodiment, the container cap 270 and the extractor cap 240 may be the same, eliminating the need for separate caps.

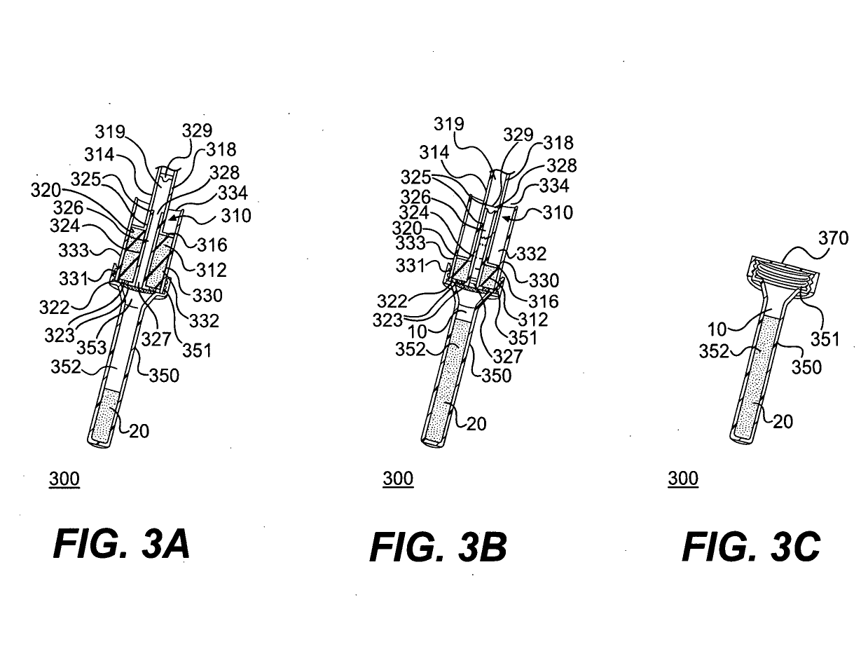

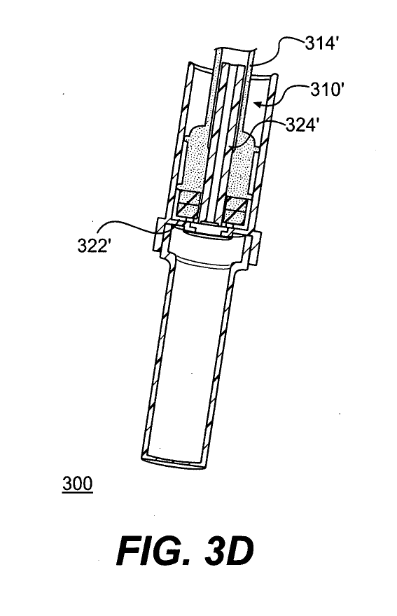

FIG. 3A illustrates yet another embodiment of the present invention. In particular, the collection system 300 includes a collector 310, an extractor 330, and container 350.

The collector 310 has an element 312 adapted to receive a sample from a source of bodily fluid. Similar to the collecting element 112 of the collector system 100 described previously, the collecting element 312 may be a treated or untreated sponge, pad, or the like, formed from an absorbent material.

The collector 310 also includes a plunger 314, which has a longitudinal handle 318 extending from a plunger end 316. Similar to the plunger handle 118 described previously, the plunger handle 318 enables a user to operate, or manipulate, the collector 310.

As further illustrated in FIG. 3A, the collector 310 has a bottom end section 322. A longitudinal stem 324 extends from the end section 322 and is partially received into an inner cavity 319 extending longitudinally through the plunger handle 318. The plunger 314 is guided over the stem 324 when the plunger moves longitudinally. The stem 324 may have annular ribs 325 which keep the stem 324 within the inner cavity 319, guide the stem 324 through the inner cavity 319, and/or help prevent fluid from escaping into the inner cavity 319.

The collecting element 312 is positioned between the plunger end 316 and the end section 322. Because the stem 324 extends between the plunger end 316 and the end section 322, the collecting element 312 has an annular shape positioned around the stem 324. Correspondingly, the plunger end 316 and the end section 322 may have annular disc-like shapes.

An overflow cavity 326 extends longitudinally through the stem 324. As described further below the overflow cavity 326 receives, through an overflow opening 327, any volume of the sample fluid that is released from the collecting element 312 but that exceeds the required amount. The overflow cavity 326 also has a valve opening 328. When the valve opening 328 remains open, the overflow cavity 326 is able to receive fluid, as the air within the overflow cavity 326 can escape through the valve opening 328 and be displaced by incoming fluid. However, a stopper 329 is fixed within the inner cavity 319 of the plunger handle 318. Therefore, when the plunger 314 moves over the stem 324 for a distance, the stopper 329 engages and closes the valve opening 328, substantially preventing any flow of fluid into the overflow cavity 326. In this way, the flow of fluid into the overflow cavity 326 depends on the position of the plunger 314 relative to the stem 324 as well as the end section 322.

Of course, alternative embodiments may employ a similar collector 310′ which does include an overflow chamber, as illustrated in FIG. 3D. In FIG. 3D, the plunger 314′ is guided over a stem 324′ which extends from an end section 322′. The stem 324′ does not have an interior overflow chamber for receiving excess sample fluid.

As illustrated in FIG. 3A, the end section 324 has two screens 323 which permit fluid communication between the collecting element 312 and the extractor 330 and the container 350. The screens 323 may be injection molded, and they may be separate pieces which are added as part of the device assembly.

The collection system 300 employs a substantially cylindrical container 350 to receive and store the sample from the collecting element 312 of the collector 310. FIG. 3A shows that the container 350 may contain a diluent 20, such as a surfactant containing solution (e.g., Tween 20) with a preservative (e.g., Chlorhexadine or Proclin 5000), with which the sample may be stored. The diameter of the container 350 may be small in order to make changes in the volume of sample 10 in the container 350 more evident. However, as shown in FIG. 3A, the cylindrical shape of the container 350 may expand in diameter to form a funnel-like shape 353, where the container 350 is joined to the extractor 330. This funnel like shape 353 helps to prevent any diluent 20 in the container 350 from entering the overflow chamber 326, as the greater diameter provides a greater volume for receiving the diluent 20 and causes the diluent 20 to rise at a slower rate.

The collector 310 is received into the extractor 330, where the collector 310 is operated to release, or express, the sample into the container 350. The extractor 330 is detachably connected to the container 350. A bottom mating section 331 of the extractor 330 fits into an upper mating section 351 of the container 350 with a substantially watertight seal. The detachable fit shown in FIG. 3A is achieved by employing screw-threads, but other techniques including, but not limited to, a snap fit, tight frictional engagement, a temporary adhesive, or the like, may be employed.

In the particular embodiment illustrated in FIG. 3A, the extractor 330 includes an extractor cavity 332 defined by a wall 333. The extractor cavity 332 has an upper opening 334, through which the collector 310 is received. The extractor cavity 332 is substantially cylindrical and is sized to receive at least the collecting element 312 and the plunger end 316. The plunger end 316 also has annular seal 320 which contacts the wall 333.

In operation, the user applies the collector 310 to collect a sample of bodily fluid in a manner similar to the application of the collector 110 described previously. After the sample is collected by the collecting element 312, the user, while holding the collector 310 by the handle 318, positions the collecting element 312 and the plunger end 316 in the extractor cavity 332 of the extractor 330.

With the collector 310 and the extractor 330 thus engaged, the user operates the handle 318 of the collector 310 to move the plunger 314 toward the container 350. Correspondingly, the plunger end 316 moves against, and applies pressure to, the collecting element 312, forcing the collecting element 312 initially into abutment with the end section 322. As shown in FIG. 3B, further downward movement of the plunger 314 handle and the plunger end 316 causes longitudinal compression of the collecting element 312 between the plunger end 316 and the end section 322. The shapes of the plunger end 316 and the end section 322 correspond with the top and bottom surfaces of the collecting element 312, so that appropriately uniform pressure can be applied on both top and bottom sides of the collecting element 312. With this compression, the size of the collecting element 312 and the volume of fluid the collecting element 312 can hold are reduced. As such, some of the sample fluid in the collecting element 312 is released. In this way, the movement of plunger 314 controls the rate of release, or expression, of the sample fluid.

When the plunger 314 is operated to release the sample from the collecting element 312, the sample passes through the screens 323 into the container 350. The seals 320 help to ensure that the sample fluid released from the collecting element 312 does not escape along the wall 333 past the plunger end 316. Advantageously, the screen 323 reduces aeration of the sample as it enters the container 350. During operation of the plunger 314, some escape of air from the system 300 may be required in order to permit the air in the container 350 to escape and be displaced by sample fluid from the collecting element 312.

The plunger 314 is operated to compress the collecting element 312 until a sufficient volume of the sample is released into the container. As FIG. 3B illustrates, the sufficient volume 10 may be substantially equal to the available volume above the diluent 20 in the container 350. However, the plunger 314 may release more than the required amount of fluid sample. In this case, the excess sample fluid enters through the overflow opening 327, as shown in FIG. 3B. In this way, the volume of sample fluid released into the container is controlled, or limited to approximately a particular volume.

The plunger 314 proceeds further downward until the stopper 329 closes the valve opening 328. The stopper 329 prevents any further flow of fluid into the overflow chamber 326 or any escape of fluid from the overflow chamber 326. At this point, a sufficient volume of sample fluid has been released from the collecting element 312.

Once the appropriate sample volume 10 is received into the container 350, the extractor 330 and the collector 310 may be removed from the container 350. As FIG. 3C shows, a container cap 370 may then be employed to seal the container 350 and protect the integrity of the sample. Once the container 350 is sealed, the sample may be stored in the container 350 and delivered for testing.

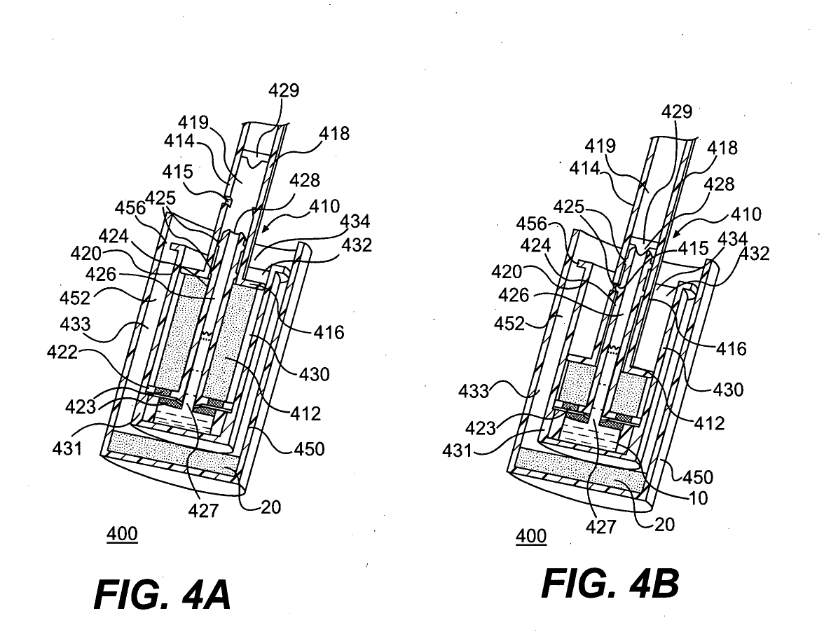

FIG. 4A illustrates a further embodiment of the present invention. In particular, the collection system 400 has a collector 410, an extractor 430, and a container 450. The collector 410 is similar to the collector 310 described previously. In summary, the collector 410 has a plunger 414 which includes a longitudinal plunger handle 418 extending from the plunger end 416. The collector 410 also has an end section 422 and a longitudinal stem 424 that extends from the end section 422 and is received into an inner cavity 419 of the plunger handle 418. A collecting element 412, which is applied to collect a sample of bodily fluid, is positioned between the plunger end 416 and the end section 422. Thus, in operation, as illustrated in FIG. 4B, the plunger 414 is guided along the stem 424 to compress the collecting element 412 against the end section 422 to release the sample fluid from the collecting element 412 through the two levels of screens 423. The screens may be injection molded, and they may be separate pieces which are added as part of the device assembly. In addition, to control the volume of sample fluid in the container 450, an overflow chamber 426 extends through the stem 424 to receive any excess sample fluid released from the collecting element 412 through an overflow opening 427. Flow into the overflow chamber 426 is controlled by a valve opening 428 and a stopper 429, as similarly described with respect to the collection system 300.

Like the collector 310, the collector 410 is received through an opening 434 into an upper extractor cavity 432 the extractor 430, where the plunger 414 may be operated to release the sample fluid from the collecting element 412. However, a notable difference between the collection system 400 and the collection system 300 described previously is that the extractor 430 and its wall 433 form an inner chamber of the container 450, while the extractor 330 is detachably connected to the container 450. Once the sample fluid is released from the collecting element 412, the collector 410 is removed, while the extractor 430 remains in place. In order to facilitate removal of the collector 430, a vent 415 is opened when the stopper 429 engages the valve opening 428 preventing further downward movement by plunger 414. The vent 415 allows air to enter the interior of the collector 410 and minimize any vacuum resistance that may act as the collector is drawn from the upper extractor cavity 432.

Moreover, as illustrated in FIG. 4B, the released fluid sample 10 in the collection system 400 is initially collected in a lower extractor cavity 431, while a diluent 20 is stored in a separate cavity 452 of the container 450. As such, the container 450, with the extractor 430 as an interior chamber, forms a “bi-level” sample collection container, or vial. However, the fluid sample 10 may be subsequently mixed with the diluent 20, as openings 456 provide fluid communication between the lower extractor cavity 431 and the cavity 452. Advantageously, the diluent 20 does not enter the overflow chamber 436, because the extractor 430 separates the diluent 20 from the overflow chamber 436, and the overflow chamber 436 is removed before any mixing occurs between the sample fluid 10 and the diluent 20.

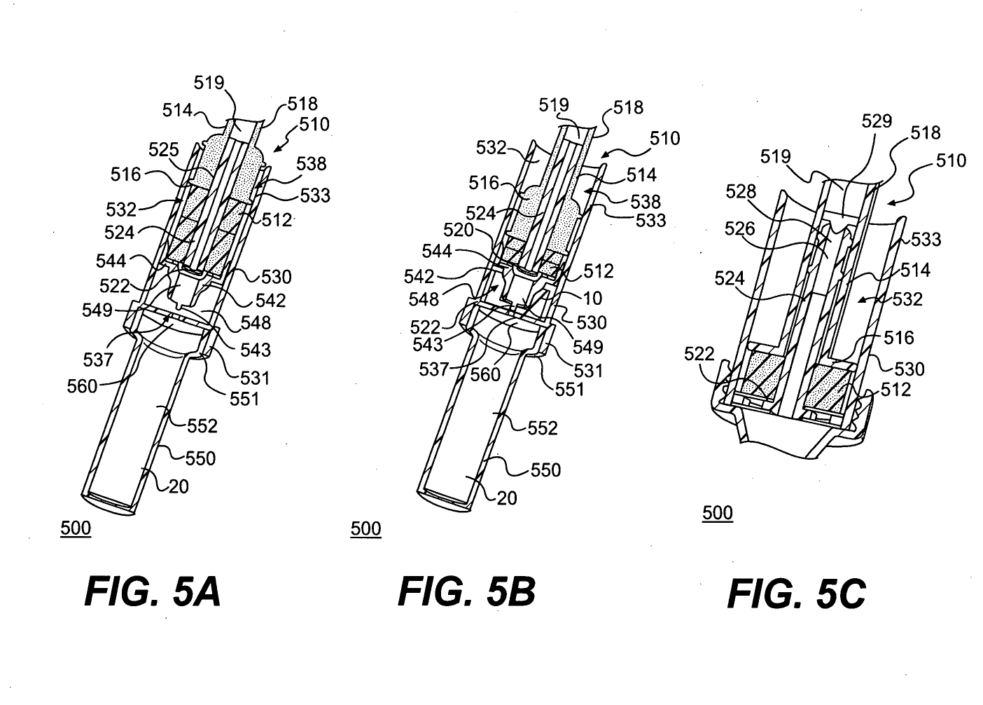

FIG. 5A illustrates yet another embodiment of the present invention. In particular, the collection system 500 has a collector 510, an extractor 530, and a container 550. Similar to collectors 310 and 410 described previously, the collector 510 has a sample collecting element 512, which is applied to a source of bodily fluid to obtain a sample for testing. The collecting element 512 may be a treated or untreated sponge, pad, or the like, formed from an absorbent material.

The collector 510 also includes a plunger 514, which has a longitudinal handle 518 extending from a plunger end 516. Similar to the plunger handle 118 described previously, the plunger handle 518 enables a user to operate, or manipulate, the collector 510.

As further illustrated in FIG. 5A, the collector 510 has a bottom end section 522. A longitudinal stem 524 extends from the end section 522 and is partially received into an inner cavity 519 extending longitudinally through the plunger handle 518. The plunger 514 is guided over the stem 524 when the plunger moves longitudinally. The stem 524 may have annular ribs 525 which keep the stem 524 within the inner cavity 519, guide the stem 524 through the inner cavity 519, and/or help prevent fluid from escaping into the inner cavity 519.

The collecting element 512 is positioned between the plunger end 516 and the end section 522. Thus, the plunger 514 may be operated to move longitudinally toward the end section 522 and compress the collecting element 512 between the plunger end 516 and the end section 522.

The extractor 530 is detachably connected to the container 550. In particular, the extractor has a bottom mating section 531 that fits over an upper mating section 551 of the container 550. The upper mating section 551 includes an opening to the interior cavity 552 of the container 550. The fit between the bottom mating section 531 and the upper mating section 551 may be achieved by techniques including, but not limited to, a snap fit, tight frictional engagement, screw-threads, or the like. The bottom mating section 531 is positioned immediately below the extractor cavity 532, and an opening 537 is positioned therebetween.

However, a breakable seal, or membrane, 560 is positioned on the container 550 over the upper mating section 551. The seal 560 may be formed from any substantially impermeable material, such as a metal foil, but must be capable of being broken, torn, or ruptured. The seal 560 may be attached to the upper mating section with an adhesive, mechanical fastening, or the like. The seal 560 keeps the container 550, which may have a measured amount of diluent, such as a surfactant containing solution (e.g., Tween 20) with a preservative (e.g., Chlorhexadine or Proclin 5000), free from contaminants until the container 550 receives the fluid sample. Moreover, because the bottom mating section 531 fits over the upper mating section 551, the seal 560 blocks fluid communication through the opening 537 and between the extractor cavity 532 and the container 550. As described further below, the seal 560 is employed to create a two-step process for releasing the fluid sample into the container 550.

As FIG. 5A shows, the extractor 530 has a penetrating structure 542 which is adapted to break the seal 560. The penetrating structure 542 divides the extractor cavity 532 into two sections, a receiving cavity 538 for receiving the collector 510 and an intermediate chamber 548 for receiving the fluid sample, as described further below. The penetrating structure 542 provides fluid communication between the receiving cavity 538 and the intermediate chamber 548 with a screen 549, which may prevent aeration.

The penetrating structure 542 is initially held in place by frictional engagement with an annular rib 544 extending inwardly from the wall 533. However, sufficient force overcomes the frictional resistance created by the annular rib 544, and the penetrating element 542 may then move longitudinally toward the opening 537 and the seal 560 that may be blocking the opening 537. The penetrating structure 542 may have a piercing element 543 to engage the seal 560 and to make the initial cut into the seal 560. Moreover, to facilitate the creation of the opening, the penetrating structure 542 may have a shape that generally tapers to a greater width, or diameter, as the penetrating structure 542 extends away from the piercing element 543, as illustrated in FIG. 5A.

In operation, a user applies the collector 510 to a source of bodily fluid to receive a sample into the collecting element 512. The collector 510 is then introduced into the extractor cavity 532 of the extractor 530. In particular, the collector is positioned in the receiving cavity 538, with the end section 522 of the collector 510 abutting the penetrating structure 542 which forms the bottom of the receiving cavity 538. Similar to embodiments previously described, the user releases, or expresses, the sample fluid by operating the plunger 514 with the plunger handle 518 to compress the collecting element 512 between the plunger end 516 and the end section 522. In the embodiment of FIG. 5B, the plunger 514 is operated to compress the collecting element 512 until an annular rib 520 abuts a part of the penetrating structure 542, which prevents further motion by the plunger 514 relative to the end section 522 abutting the penetrating structure 542.

The sample fluid is released through the screen 549 in the penetrating structure 542. However, due to the seal 560 blocking fluid communication with the container 550, the sample fluid is not immediately released into the container 550. Rather, the sample fluid is released into an intermediate chamber 548, which is defined in the extractor cavity 532 by the seal 560 and the penetrating section 542.

Once plunger 514 has been operated to release the sample fluid into the intermediate chamber 548 and can no longer move relative to the end section 522, the user operates the plunger handle 518 to push the collector 510 against the penetrating structure 542. Since the plunger 514 can no longer move relative to the rest of the collector 510, the entire collector 510 moves with the plunger 514. With the application of sufficient force, the penetrating element 542 overcomes the frictional engagement with the annular rib 544 and is pushed toward the seal 560. With the motion of the penetrating element 542, the piercing element 543 engages the seal 560 and ruptures the seal 560. The collector 510 and the penetrating element 542 proceed further through the seal 560 to create a greater opening through the seal. Once the seal 560 is broken, further movement of the collector 510 into the extractor 530 pushes the released sample in the intermediate chamber 548 through the opening 537 and into the container 550. Once the sample fluid is introduced into the container 550, the extractor 530 and the collector 510 may be removed from the top of the container 550, and the container 550 may be capped and delivered for testing.

Therefore, the collection system 500 illustrated by FIGS. 5A-B employs the seal 560 to create a two-step system. In the first step, the plunger 514 is operated with the plunger handle to release the sample fluid from the collecting element 512 into the intermediate chamber 548. In the second step, the plunger handle 518 is operated to move the entire collector 510 and the penetrating structure 542, to break the seal 560 over the container 550 and introduce the sample fluid into the container 550.

In its initial position, the penetrating structure 542 defines an intermediate chamber 548 with a predetermined volume. In order to ensure that no more than this predetermined volume is released into the container 550, an overflow chamber 526 may be optionally positioned, as shown in FIG. 5C, within the stem 524, in order to receive any excess sample fluid. Similar to the collection systems 300 and 400 previously described, the overflow chamber 526 has an overflow opening 528. Flow into the overflow chamber 526 is controlled by a valve opening 528 on the other end of the stem 524 and a stopper 529 positioned in the inner cavity of 519 of the plunger 514.

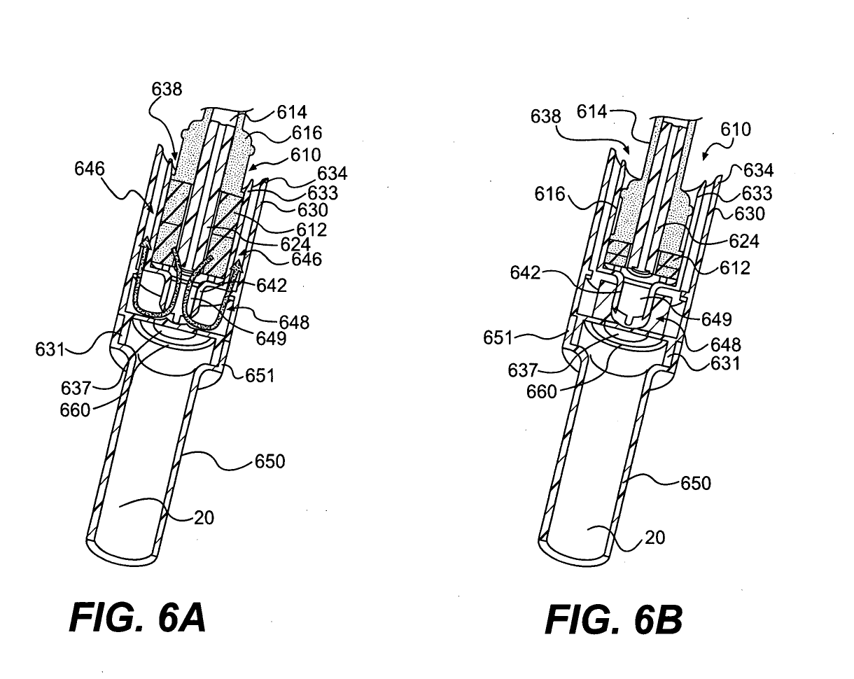

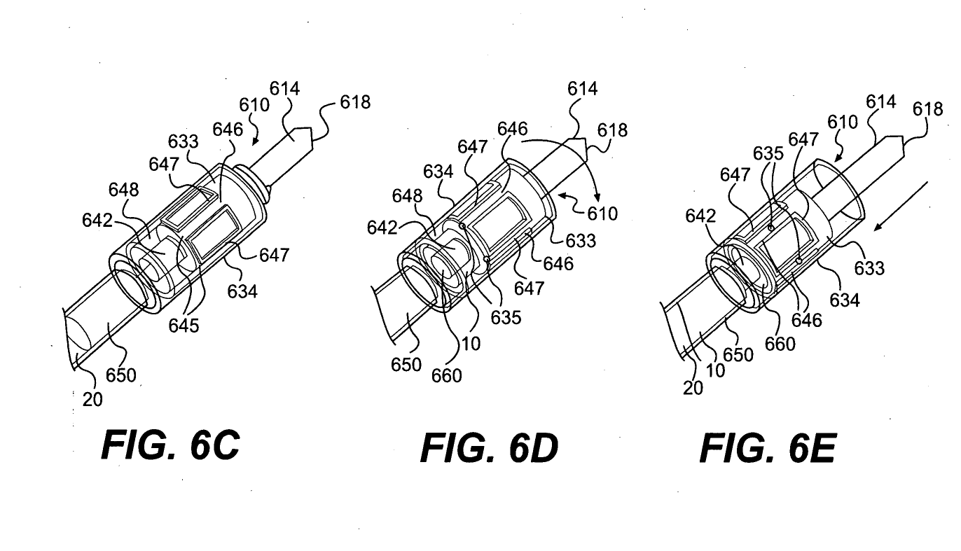

Alternatively, referring to FIG. 6A, a collector system 600 may employ overflow chambers 646 that are positioned within an extractor 630, rather than an interior stem 625 of a collector 610. As shown in FIG. 6A, the collection system 600 is similar in many respects to the collection system 500 described previously. However, the extractor 630 has two walls, an interior wall 633 and an exterior wall 634. The interior wall 633 extends upwardly from the penetrating structure 642 to define a receiving cavity 638 which receives the collector 610. The interior wall 633 and the receiving cavity are positioned within the exterior wall 634 with a space between the interior wall 633 and the exterior wall 634. As shown in FIG. 6C, the overflow chambers 646 are formed by chamber walls 647 between the interior wall 633 and the exterior wall 644. In particular, the chamber walls 647 may extend radially outward from the outer surface of the interior wall 633. The chamber walls 647 form a plurality of elongate overflow chambers 646 that are distributed circumferentially around the cylindrical outer surface of the inner wall 633. The overflow chambers 646 are in fluid communication with the intermediate chamber 648 through overflow openings 645, so that excess sample fluid introduced into the intermediate chamber 648 may be received into the overflow chambers 646. Each overflow chamber 646 extends upwardly from an overflow opening 645 at the penetrating element 642. Extending inwardly from the exterior wall 644 are valve closures 635, which may be tab-like structures. In introduce the sample fluid into the container 650. As FIG. 6E further illustrates, the valve closures 635 are not only sized to seal the overflow openings 645, but are sized to allow the valve closures 635 to move through the overflow chambers 646 so that the interior wall 633 may move with the penetrating structure 642 as the collector 610 pushes the penetrating structure 642 forward. As such, any excess sample in the overflow chambers 646 is pushed by the valve closures 635 along through the overflow chambers 646 toward the receiving opening 634. Once the sample fluid is introduced into the container 650, the extractor 630 and the collector 610 may be removed from the top of the container 650, and the container 650 may be capped and delivered for testing.

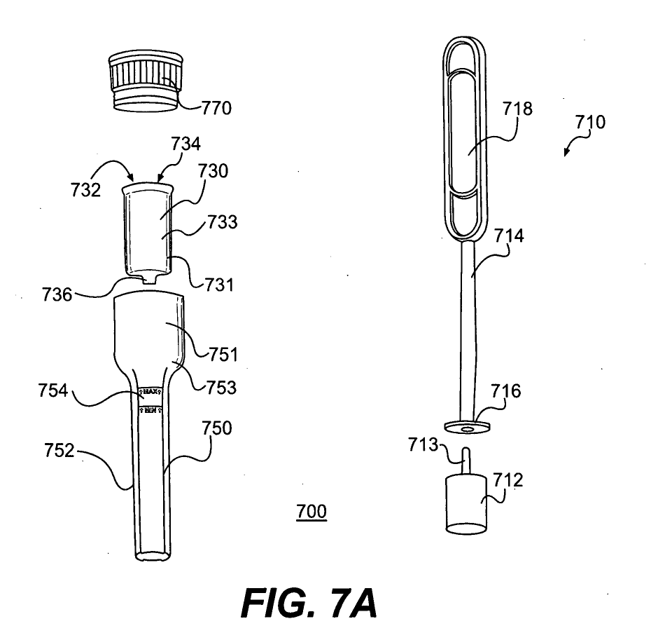

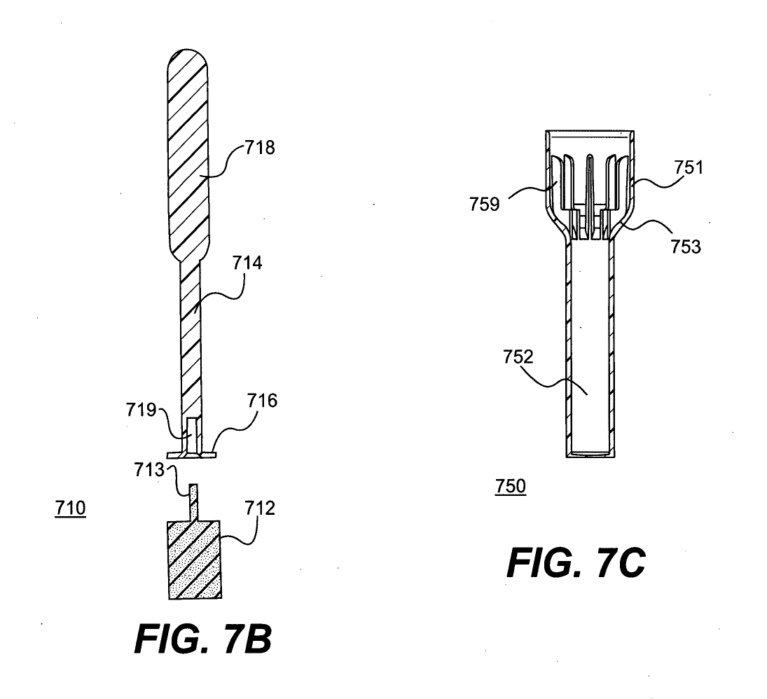

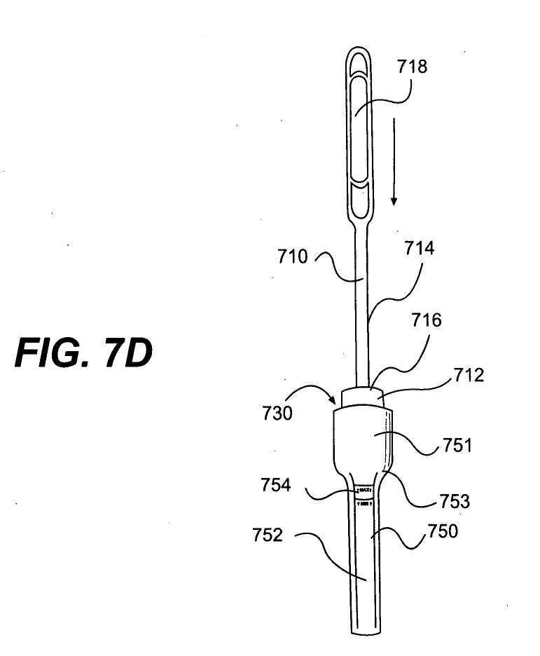

FIG. 7A illustrates an exploded view of yet another embodiment. In particular, the collection system 700 includes a collector 710, an extractor 730, a container 750, and a container cap 770.

The collector 710 employs an element 712 adapted to receive a sample from a source of bodily fluid. Like the collecting element 112 of collection system 100 described previously, the collecting element 712 may be a pad, sponge, or the like, formed from an absorbent material. The absorbent material may include natural occurring absorbent materials, such as, but not limited to, cotton or cellulose sponges as well as synthetic fibers, such as, but not limited to, polyesters. Thus, when the collecting element 712 is applied to, or placed into contact with, a source of fluid, it absorbs some of the fluid from the source. In addition, the collecting element 712 may be treated to optimize recovery of analytes from the sample as also discussed herein. The collecting element 712 is initially sized so that a sufficient volume of the sample fluid may be absorbed from the fluid source.

The collecting element 712 generally holds the sample until the collecting element 712 is manipulated to release, or express, the sample. For example, the sample held by the collecting element 712 may be released from the absorbent material by compressing the collecting element 712, thus reducing the order to open and close the overflow openings 645, the receiving cavity 638 defined by the interior walls 633 may be rotated relative to the exterior wall 644. When the receiving cavity 638 is properly aligned with respect to the exterior wall 644, the valve closures 635 close all of the overflow openings 645.

In operation, the collector 610, which has been applied to collect a sample of bodily fluid, is introduced into the receiving cavity 648 of the extractor 630. The collector 610 is initially oriented so that the overflow openings 645 are not aligned with the valve closures 635 and are in fluid communication with the intermediate chamber 648. Structures, such as grooves and corresponding tab-like structures, may be employed between the extractor 630 and the collector 610 to ensure proper initial alignment.

The plunger 614 is then operated with the plunger handle to release the sample fluid from the collecting element 612 into the intermediate chamber 648, as illustrated in FIG. 6C. Any excess sample fluid that cannot be accommodated by the volume of the intermediate chamber 648 is received by the overflow chambers 646, as shown in FIG. 6A.

Once the plunger 614 has been operated to release the sample fluid into the intermediate chamber 648 and can no longer move relative to the end section 622, the user closes the overflow openings 646 by rotating the interior wall 633 and aligning the overflow openings 646 with the valve closures 635, as illustrated in FIG. 6D. The collector 610 may engage the interior wall 633 or the penetrating structure 642 in a manner that allows the plunger handle 618 to be operated to accomplish this relative rotation. Structures, such as grooves and corresponding tab-like structures, may also be employed to guide the rotation of the interior wall 633. Preferably, the rotation requires a quarter-turn to close the overflow openings 645.

Once the overflow openings 645 are closed, the plunger handle 618 may then be operated, as shown in FIG. 6E, to move the entire collector 610 and the penetrating structure 642, to break the seal 660 over the container 650 and volume of the collecting element 712 and its ability to hold the sample. As shown in FIG. 7A, the collecting element 712 may be substantially cylindrical in shape, but the collecting element 712 is not limited to this particular shape. As described further below, the shape of collecting element 712 generally corresponds to the shape of the extractor 730 so that the collector 710 may be employed with the extractor 730 as well as the container 750.

As further illustrated in FIG. 7A, the collecting element 712 is attached to an end 716 of a plunger 714. The various techniques that may be employed to attach the collecting element 712 to the plunger end 716 include, but are not limited to, the use of adhesives, chemical bonding, fasteners, mechanical joining, or the like, or any combination thereof. The end 716 of the plunger 714 may be substantially disc-shaped to correspond with the substantially cylindrical shape of the collecting element 712. However, the plunger end 716 is not limited to this particular shape. In general, the plunger end 716 is shaped to enable the plunger 714 to be operated to apply pressure to at least a portion of a side, e.g. the top side, of the collecting element 712 and more preferably to an entire side, as described further below.

The plunger 714 also has a longitudinal handle 718 which extends from the plunger end 716. The plunger handle 718 enables a user to operate, or manipulate, the collector 710 to cause the sample to be released, or expressed, from the collecting element 712, as described further below.

FIG. 7B shows a cross-sectional view of the collector 710. In particular, an inner passage 719 extends through the center of the plunger end 716 into a portion of the handle 718. Advantageously, an indicator wick 713 may be inserted into the inner passage 719. The indicator wick 713 may be another treated or untreated fiber material that is in contact with, or extends from, the collecting element 712. The indicator wick 713 exhibits a physical change when the collecting element 712 is sufficiently saturated, i.e., absorbs sufficient sample fluid, to allow the sample fluid to wet the indicator wick 713. In other words, when the indicator wick 713 exhibits the physical change, the user is able to determine that the collecting element 712 has collected enough sample fluid, thereby minimizing errors in the collection of the sample fluid. For example, the physical change may be a color change that is driven by moisture, dye migration, or a pH change caused by the sample fluid. The plunger handle 118, or an appropriate portion thereof, may be translucent to enable a user to observe the physical change of the indicator wick 713 within the inner passage 719. It is contemplated that other indicators may be employed to indicate the collection of a sufficient volume of sample fluid with the embodiments described herein. For example, the collecting element 712 itself may exhibit an observable physical change when it absorbs sufficient sample fluid.

Referring again to FIG. 7A, the collection system 700 employs the tube-like container 750 to receive and store the sample from the collecting element 712 of the collector 710. The sample is received into a lower receiving portion 752 of the container 750. The receiving section 752 is relatively narrow in order to make changes in the volume of sample in the container 750 more evident. As described in detail herein, the container 750 may contain a diluent, such as a surfactant containing solution with a preservative, with which the sample may be stored.

As shown in FIG. 7A, the narrow shape of receiving section 752 may expand to form a funnel-like shape 753, which defines a transition to a larger cup-like upper mating section 751. The upper mating section 751 receives the extractor 730, where the collector 710 is operated to release, or express, the sample into the container 750. The extractor 730 may be removed from the container 750, but in alternative embodiments, the extractor may be integrally joined or formed with the container 750. The extractor 730 has a wall 733 that defines a barrel-like cup with an extractor cavity 732. The extractor cavity 732 has an upper opening 734, through which the collector 710 is received. The extractor 730 also includes an nozzle-like bottom opening 736 through which the sample passes into the container 750. The shape and dimension of the opening 736 may provide valve-like functionality that facilitates flow from the extractor 730 into the container 750 but minimizes the flow of fluid in the opposite direction.

The extractor cavity 732 is sized to receive at least the collecting element 712 and the plunger end 716. The dimensions of the extractor cavity 732 also correspond with the disc-shaped plunger end 716, so that a seal is formed between the plunger end 716 and the extractor wall 733. The seal allows the sample to be expressed from the collecting element 712 with downward movement of the plunger 714 within the extractor 730, as described further below.

As FIG. 7C illustrates, the upper mating section 751 of the container 750 includes a series of ribs 759 that extend inwardly and longitudinally along the interior of the mating section 751. The ribs 759 receive the bottom mating section 731 of the extractor 730 in tight frictional engagement and position the extractor 730 centrally within the upper mating section 751. It is contemplated that the detachable fit between the extractor 730 and the container 750 may be alternatively achieved by employing screw-threads, a snap fit, a temporary adhesive, or the like.

Preferably, the extractor 730 is also dimensioned so that when it is positioned within the container 750, the cap 770 may be placed over the container 750. In this way, the extractor 730 may be conveniently enclosed in the container 750 during packaging and delivery to the user, minimizing the chances that the extractor 730 may become contaminated or misplaced.

In operation, the user applies the collector 710 to collect a sample of bodily fluid in a manner similar to the application of the collectors described previously. Sample fluid is absorbed by the collector 710 until the collecting element 712 is saturated. In this embodiment, once the collecting element 712 is saturated, the indicator wick 713 exhibits a physical change, such as a color change in response to contact with the sample. The physical change informs the user that the collecting element 712 has absorbed a minimum amount of oral fluid and that the collector may be removed from the mouth.

After the sample is collected by the collecting element 712, the user, while holding the collector 710 by the handle 718, positions the collecting element 712 and the plunger end 716 in the extractor cavity 732 of the extractor 730. With the collector 710 and the extractor 730 thus engaged, the user operates the handle 718 of the collector 710 to move the plunger 714, i.e., downwardly, toward the container 750. FIG. 7D illustrates the collector 710 as it is being introduced into the extractor 730 and container 750. Correspondingly, the plunger end 716 moves against, and applies pressure to, the collecting element 712. Downward movement of the plunger 714 handle and the plunger end 716 causes longitudinal compression of the collecting element 712 between the plunger end 716 and bottom portion of the extractor wall 733. The shapes of the plunger end 716 and bottom portion of the extractor wall 733 correspond with the top and bottom surfaces of the collecting element 712 to facilitate application of pressure thereto, such as, for example, to apply uniform pressure on both top and bottom sides of the collecting element 712. With this compression, the size of the collecting element 712 and the volume of fluid the collecting element 712 can hold are reduced. As such, some of the sample fluid in the collecting element 712 is released. In this way, the movement of plunger 714 controls the rate of release, or expression, of the sample fluid.

The plunger 714 is operated to compress the collecting element 712 until a sufficient volume of the sample is released into the container. A volume indicator 754, such as a mark or window, on the container 750 may be employed to alert the user when enough of the sample has been released. The volume indicator 754 may include markings indicating both a minimum amount and a maximum amount of sample that should be provided. For example, the top of the sample in the container 750 should be between the minimum and maximum amount markings.

Once the appropriate sample volume is received into the container 750, the extractor 730 and the collector 710 may be removed from the container 750. The engagement or fit between the plunger 714 and the extractor 730 facilitates removal of the extractor 730 and the collector 710. The container cap 770 may then be employed to seal the container 750 and protect the integrity of the sample. Once the container 750 is sealed, the sample may be stored in the container 750 and delivered for testing. As described above, the extractor 730 may be enclosed within the capped container 750, so in alternative embodiments, the extractor 730 is not removed from the container 750. In these alternative embodiments, a tighter fit or fixed engagement between the ribs 759 and the extractor 730 may be required.

As described previously, bodily fluid samples collected by embodiments according to aspects of the present invention may include saliva, or oral fluid. Accordingly, a further aspect of the present invention relates to a method of collecting an oral fluid specimen from an oral cavity for testing. While the method is preferably designed to obtain oral fluid samples to test for drugs of abuse in human subjects, the method may be used to obtain oral fluid sample from humans for other purposes or to obtain oral fluid samples from animals.

As also described previously, collectors in embodiments according to aspects of the present invention may employ a collecting element that is treated to optimize recovery of analytes from the sample. Therefore, according to an embodiment, a compressible, detergent-treated collecting element is inserted into the oral cavity of the subjects mouth. The collecting element is brought into contact with oral fluid within the oral cavity for a sufficient time to collect an oral fluid sample. This is done without masticating the collecting element. Once the oral fluid sample is collected, the collecting element is removed from the oral cavity. The fluid sample may then be released, or expressed, from the collecting element into a container containing a preservative in a manner employing the systems and devices described previously. Alternatively, the collecting element itself may be placed in a preservative solution for later testing of the oral fluid. Thus, it is understood that while the treatments described herein may be employed with the systems and devices described previously, they may be applied more broadly to any system or device for collecting samples of fluid.

Various types collecting elements exist, including those employed with the collectors described above. The collecting element is not limited to any particular material as long as the material used absorbs oral fluids and may be compressed to express the sample from the collecting element. The collecting element is made of an absorbent material which can be effectively placed into the oral cavity. A plastic or carbohydrate material such as cellulose can be used as the absorbent material. However, cellulose materials are preferred. Ultracell from North Slatington, Conn. provided the cellulose material used as the collecting element in the examples described below.

The collecting element may be any size or shape that fits comfortably into the mouth of the subject from whom the oral fluid sample is being obtained and that collects a sufficient amount of sample for the testing required. The maximum volume of sample that is collected will be controlled by the capacity of the collection material. An example of a collecting element is described in U.S. Pat. No. 5,103,836. As described below, a donut-shaped collecting element may also be used. Oral fluid collectors having such annular collecting elements are described in published U.S. Patent Application Publication No. 2003/0064526 A1.

The collecting element may be pre-treated with a non-ionic detergent using any means known in the art. For example, the non-ionic detergent may be applied to the collecting element by dipping the collecting element into the detergent solution so that the solution is absorbed into and onto the collecting element, removing the collecting element from the solution and allowing the collecting element to dry. Typically, the collecting element is dipped into the detergent solution at concentrations ranging from 0.1 to 1% using a sufficient amount of solution to completely saturate the collecting element. Alternatively, the detergent solution may be sprayed onto the collecting element to saturate the collecting element. Excess liquid is shaken off and the collecting element is placed into a forced air, convection drying oven at 50° C. for 2 hours. After drying, there will be a formed collecting element pre-treated with the detergent.

Preferably, any treatment applied to the collecting element is food grade and has no objectionable taste when the collecting element is administered orally. For example, the collecting element may be tasteless to the user or may be further treated with flavoring aids to make the taste more pleasant to the user.

The collecting element, with or without a holder, is brought into contact with oral fluid inside the subject's oral cavity. The collecting element may be inserted in those areas where oral fluid is excreted and/or collects in the oral cavity. Preferably, the collecting element is placed between the cheek and gum line in the subject's mouth and allowed to collect oral fluid while the device is stationary or preferably the device is moved around the mouth to facilitate the collection. The collecting element is left in contact with the oral fluid for a time sufficient to absorb enough oral fluid to fill the collecting element. Typically, the collecting element is placed in contact with the oral fluid for about 30 seconds to about 6 minutes, preferably between about 2 and about 5 minutes.

After the oral fluid sample is collected, the collecting element is removed from the subject's oral cavity. The oral fluid sample may be expressed from the collecting element by means known in the art such as by compressing or squeezing the collecting element or by centrifuging the collecting element. The expressed oral fluid sample may then be analyzed for an analyte of interest.

As an alternative to expressing and then analyzing the oral fluid sample, the collecting element containing the oral fluid or the expressed oral fluid sample may also be preserved in a preservative solution for later analysis, as previously described. As is known in the art, the preservative solution acts to inhibit enzymatic activity which can be responsible for the destruction of analytes of interest or can function as an anti-microbial agent. Compounds contemplated for use as a preservative include antibacterial agents, anti-fungal agents, bacteriostatic agents, fungistatic agents, and enzyme inhibitors. As an antibacterial agent, it is preferred to use chlorhexidine. Alternatively, Proclin 5000 may be employed.

In a preferred embodiment, oral fluid samples collected according to the invention are used in drugs of abuse testing. For example, the oral fluid samples may be used to test for marijuana (THC), nicotine (continine), cocaine metabolite (benzoylecgonine), opiates (morphine, 6-acetylmorphine, and codeine), phencyclidine, and amphetamines (amphetamine and methamphetamine). Assays and testing methods for such drugs of abuse using oral fluid samples are known in the art. See, for example, E. J. Cone et al., Oral Fluid Testing for Drugs of Abuse: Positive Prevalence Rates by Intercept Immunoassay Screening and GC-MS-MS Confirmation and Suggested Cutoff Concentrations, J. Analytical. Toxicology, vol. 26, p. 541-6, 2002.

The following examples illustrate various aspects and advantages of the employing various materials and treatments for the collecting element. In particular, examples demonstrate the recovery of THC which is a difficult drug to extract from various collecting element materials.

### EXAMPLE 1

### Polyolefin Fiber Collecting Element Material with and without Nonionic Surfactant Pretreatments

The following tests evaluate the recovery of Amphetamine, Methamphetamine, PCP, Benzoylecgonine (BE), Morphine, and THC from untreated polyolefin fiber collecting elements and polyolefin fiber collecting elements pretreated with 1 or 2 mL of a 0.75% nonionic surfactant (PEG 400 monooleate) in water solution.

To conduct the drug recovery experiments, freshly collected (same day) human oral fluid was spiked with a drug standard to a concentration of 200 ng/mL Amphetamine and Methamphetamine, 160 ng/mL Morphine, 32 ng/mL BE, 40 ng/mL PCP, and 8 ng/mL THC. Polyolefin fiber collecting elements used in this experiment are cylindrically shaped with dimensions of OD-12.33 mm, length of 29.99 mm, and a capacity of approximately 3.2 mL (from Filtrona Fibertec, Va.). The polyolefin fiber collecting elements were left untreated or were pretreated by saturating the collecting elements with 1 mL (31% of collecting element capacity) or 2 mL (63% of collecting element capacity) of a 0.75% PEG 400 monooleate in water solution and allowing them to dry overnight at 37° C. 1 mL (31% of collecting element capacity) of drug-spiked oral fluid was added to the collecting elements. The oral fluid containing collecting elements were placed in polypropylene vials and centrifuged (for 10 min at 3000 RPM) into another polypropylene tube. 100 μL of the resulting centrifugate liquid was analyzed by LC/MS/MS. As a control, 100 μL of the original drug-spiked oral fluid was also analyzed. Just prior to analysis, a fixed amount of deuterated drug was added to all the samples to serve as internal standard to correct for any extraction related losses. The concentrations of drug analytes found in the sample centrifuged off the treated polyolefin collecting element were compared to the concentrations of drug analytes found in the saliva. This ratio was expressed as a percentage recovery of drug from the collecting element material.

### EXAMPLE 1

### Results

| | Volume of 0.75% PEG | |
|---|---|---|
| Analyte | Pretreatment (mL) | % Recovery |
| Morphine | 0 | 97 |
| | 1 | 92 |
| | 2 | 89 |
| Amphetamine | 0 | 88 |
| | 1 | 92 |
| | 2 | 92 |
| Methamphetamine | 0 | 96 |
| | 1 | 86 |
| | 2 | 84 |
| BZE | 0 | 97 |
| | 1 | 96 |
| | 2 | 93 |
| PCP | 0 | 16 |
| | 1 | 21 |
| | 2 | 33 |
| THC | 0 | 15 |
| | 1 | 66 |
| | 2 | 107 |

The above experiment yielded drug recoveries between 84% and 97% for Morphine, Amphetamine, Methamphetamine, and BE from the treated and untreated polyolefin fiber collecting element conditions. The PCP drug recovery increased from 16% to 33% with 2 mL of 0.75% PEG 400 monooleate collecting element pretreatment. THC drug recovery increased from 15% to 107% with 2 mL of 0.75% PEG 400 monooleate collecting element pretreatment.

### EXAMPLE 2

### Polyolefin Fiber Collecting Element Material with 0.75% Nonionic Surfactant Pretreatment Tested with Ten Individual's Oral Fluid

The following tests evaluate the recovery of Amphetamine, Methamphetamine, PCP, Benzoylecgonine (BE), Morphine, and THC from polyolefin fiber collecting elements pretreated with a 0.75% nonionic surfactant (PEG 400) in water solution for ten individual's oral fluid.

To conduct the drug recovery experiments, freshly collected (same day) human whole oral fluid samples from 10 individuals were spiked with a drug standard to a concentration of 125 ng/mL Amphetamine and Methamphetamine, 100 ng/mL Morphine, 20 ng/mL BE, and 10 ng/mL PCP and THC. Polyolefin fiber collecting elements used in this experiment are cylinder shaped with dimensions of OD-12.43 mm, ID-1.5 mm, length of 11.96 mm, and a capacity of approximately 1.3 mL (from Filtrona Fibertec, Va.). The polyolefin fiber collecting elements were pretreated by saturating the collecting elements with 1.5 mL (114% of collecting element capacity) of a 0.75% PEG 400 in water solution and allowing them to dry overnight at 37° C. The collecting elements were saturated with 1.5 mL (114% of collecting element capacity) with each individual's drug-spiked oral fluid. The oral fluid containing collecting elements were placed in polypropylene vials and centrifuged (for 10 min at 3000 RPM) into another polypropylene tube. 100 μL of the resulting centrifugate liquid was analyzed by LC/MS/MS. As a control, 100 μL of the original drug-spiked oral fluid was also analyzed. Just prior to analysis, a fixed amount of deuterated drug was added to all the samples to serve as internal standard to correct for any extraction related losses. The concentrations of drug found in the sample centrifuged off the treated polyolefin collecting element were compared to the concentrations of drug found in the oral fluid. This ratio was expressed as a percentage recovery of drug from the collecting element material.

### EXAMPLE 2

### Results

| | Minimum % | Maximum % | Average % |
|---|---|---|---|
| | Recovery for | Recovery for | Recovery for 10 |
| Analyte | 10 Individuals | 10 Individuals | Individuals |
| Morphine | 99 | 104 | 102 |
| Amphetamine | 94 | 105 | 101 |
| Methamphetamine | 98 | 109 | 101 |
| BE | 99 | 104 | 101 |
| PCP | 68 | 97 | 79 |
| THC | 71 | 133 | 92 |

The above experiment yielded average drug recoveries of 102%, 101%, 101%, 101%, 79%, and 92% for Morphine, Amphetamine, Methamphetamine, BE, PCP and THC respectively from the polyolefin fiber collecting element pretreated with a 0.75% PEG 400 in water solution.

### EXAMPLE 3

### Oral Fluid Recovery from Untreated Collecting Elements Tested on Human Volunteers

The following tests evaluate the amount of oral fluid recovered when expressed off the collecting elements into the container using the extractor. Polyolefin fiber collecting elements used in this experiment are cylinder shaped with dimensions of OD-12.22 mm, length of 16.14 mm, and a capacity of approximately 1.8 mL (from Filtrona Fibertec, Va.). Fiber wicks used in this experiment are cylinder shaped with dimensions of OD-2.05 mm and cut to a length of 15 mm. The wicks were marked at 10 mm with marker ink that bleeds when wet. The wicks were placed into the polypropylene sticks and the polyolefin fiber collecting elements were glued onto the polypropylene sticks containing the indicator wicks. Human volunteers were instructed to collect oral fluid via the following method: swab 3 times around both cheeks then hold under the tongue for 2 minutes and repeat process until the indicator wick changed color. Timing of collection began when volunteer began swabbing and ended when the indicator wick changed color. The oral fluid extractor and container assembly was weighed before and after the collecting elements were expressed. The difference in the weights determined the volume of oral fluid expressed off the collecting element. The ratio of oral fluid volume collected on the collecting element to the volume of oral fluid expressed off the collecting element is shown as the percentage recovery of oral fluid.

### EXAMPLE 3

### Results

| | | % Oral |
|---|---|---|
| | Oral fluid | fluid |
| | Volume | Recovered |
| | Collected | after |
| Sample # | (mL) | Expression |
| 1 | 1.3 | 85 |
| 2 | 1.3 | 77 |
| 3 | 1.1 | 82 |
| 4 | 1.3 | 69 |
| 5 | 0.8 | 75 |
| 6 | 1.1 | 73 |
| 7 | 1.1 | 82 |
| 8 | 1.1 | 91 |

The amount of oral fluid recovered from the collecting element ranged from 69% to 91% with an average of 79%.

### EXAMPLE 4

### Sample Cotton Collecting Element Material Vs. Compressible Cellulose Material with No Detergent Treatment

The following tests evaluate the recovery of THC from compressible type cellulose collecting elements and a cotton collecting element with no pretreatment of the materials to show what the lack of any pretreatment has on THC recovery.

To conduct the THC recovery experiments, freshly collected (same day) human whole saliva was spiked with a THC standard to a concentration of 15 ng/mL. 400 μL of the THC-spiked whole saliva was applied to each of the collecting elements. Compressible type cellulose collecting elements used in this experiment are donut shaped with dimensions of OD-1.3 cm, ID-0.5 cm and thickness of 0.9 mm (from Ultracell, Conn.). The cotton collecting elements were similar to those disclosed in U.S. Pat. No. 5,103,836. The saliva containing collecting elements were placed in polypropylene vials containing 800 μL of a preservative solution for a period of 60 minutes. The preservative solution is comprised of chlorhexidine, Tween 20 and a blue dye. The preservative solution was centrifuged (for 10 min at 3000 RPM) into another polypropylene tube. 400 μL of the resulting centrifugate liquid was analyzed by LC/MS % MS. As a control, 133 μL of the original THC-spiked saliva was also analyzed. Just prior to analysis, a fixed amount of d3-THC was added to all the samples to serve as internal standard to correct for any extraction related losses. The concentration of THC found in the sample extracted into the preservative solution was compared to the concentration of THC found in the saliva. This ratio was expressed as a percentage recovery of THC from the collecting element material.

### EXAMPLE 4

### Results

| Sample | Value | Mean | % Recovery |
|---|---|---|---|
| saliva-1 | 6.25 | 6.14 | 100 |
| saliva-2 | 6.02 | | |
| cotton-1 | 1.19 | 1.34 | 22 |
| cotton-2 | 1.48 | | |
| cellulose-1 | 3.76 | 3.79 | 62 |
| cellulose-2 | 3.82 | | |

The above experiment yielded a THC recovery of 22% from the cotton collecting element material and 62% recovery from the compressible cellulose collecting element material when neither of the collecting element materials was pretreated with any detergents.

### EXAMPLE 5

### Cotton Collecting Element Material Pre-Treated with Tween-20 vs. Cellulose Collecting Element Material with and without Pre-Treatment with Tween-20

The following tests evaluate the recovery of THC from the cotton collecting element material pre-treated with Tween-20 and compressible cellulose material, with and without pre-treatment of Tween-20 at concentrations of 0.1, 0.25 and 0.5%. Experiment was carried out as in the previous example except the collecting elements were pre-treated with Tween-20: The cotton collecting elements were pre-treated with 700 μL and the cellulose collecting elements were pre-treated with 600 μL of 0.1%, 0.25% and 0.5% Tween-20 solution. All the collecting elements were allowed to dry overnight at 38° C. Calculations were performed as in the previous example.

### EXAMPLE 5

### Results

| Sample | Value | Mean | % Recovery |
|---|---|---|---|
| Saliva-1 | 5.58 | 5.67 | 100 |
| Saliva-2 | 5.76 | | |
| 0.1% Tween cellulose-1 | 4.09 | 4.03 | 71 |
| 0.1% Tween celluose-2 | 3.97 | | |
| 0.25% Tween cellulose-1 | 4.18 | 4.29 | 76 |
| 0.25% Tween cellulose-2 | 4.39 | | |
| 0.5% Tween cellulose-1 | 4.3 | 4.41 | 78 |
| 0.5% Tween cellulose-2 | 4.52 | | |
| 0.1% Tween cotton-1 | 1.87 | 1.74 | 31 |
| 0.1% Tween cotton-2 | 1.6 | | |
| 0.25% Tween cotton-1 | 1.73 | 2.06 | 36 |
| 0.25% Tween cotton-2 | 2.38 | | |
| 0.5% Tween cotton-1 | 2.28 | 2.35 | 41 |
| 0.5% Tween cotton-2 | 2.42 | | |

The above experiment demonstrated an increase in THC recovery in both the cotton and Ultracell collecting element materials with the pretreatment of Tween-20. In addition, as the concentration of Tween-20 increased, so did the THC recovery in both collecting element materials.

### EXAMPLE 6

### Expanded Range of Tween Concentrations Using Cellulose Collecting Elements

The following tests evaluate THC recovery from compressible cellulose collecting element material pre-treated with Tween-20 at concentrations of 0.05%, 0.1%, 0.25%, 0.5%, 1.0%

The experiment was carried out as in the previous examples with the following exceptions: 1) only the Ultracell cellulose collecting elements were pretreated with the different concentrations of Tween-20; and 2) samples were run in replicates of 5.

### EXAMPLE 6

### Results

| Sample Name | THC (ng) | Mean | % Recovery |
|---|---|---|---|
| saliva-1 | 5.09 | 6.1 | 100 |
| saliva-2 | 7.1 | | |
| 0.05% Tween-cell-1 | 3.71 | 3.7 | 61 |
| 0.05% Tween-cell-2 | 3.65 | | |
| 0.05% Tween-cell-3 | 3.56 | | |
| 0.05% Tween-cell-4 | 3.81 | | |
| 0.05% Tween-cell-5 | 3.75 | | |
| 0.1% Tween-cell-1 | 3.85 | 3.91 | 64 |
| 0.1% Tween-cell-2 | 3.37 | | |
| 0.1% Tween-cell-3 | 3.9 | | |
| 0.1% Tween-cell-4 | 4.15 | | |
| 0.1% Tween-cell-5 | 4.3 | | |
| 0.25% Tween-cell-1 | 4.66 | 4.57 | 75 |
| 0.25% Tween-cell-2 | 2.66* | | |
| 0.25% Tween-cell-3 | 4.57 | | |
| 0.25% Tween-cell-4 | 4.59 | | |
| 0.25% Tween-cell-5 | 4.44 | | |
| 0.5% Tween-cell-1 | 4.76 | 4.68 | 77 |
| 0.5% Tween-cell-2 | 4.66 | | |
| 0.5% Tween-cell-3 | 4.83 | | |
| 0.5% Tween-cell-4 | 5.03 | | |
| 0.5% Tween-cell-5 | 4.14 | | |
| 1.0% Tween-cell-1 | 4.69 | 4.97 | 82 |
| 1.0% Tween-cell-2 | 4.61 | | |
| 1.0% Tween-cell-3 | 4.71 | | |
| 1.0% Tween-cell-4 | 5.2 | | |
| 1.0% Tween-cell-5 | 5.66 | | |

The above experiment demonstrated an increase in THC recovery up to 1% Tween which appeared to level off as the concentration approached 1%.

### EXAMPLE 7

### Compare Saliva Centrifuged Off the Collecting Element into Preservative to Collecting Elements Soaked in Preservative

The following tests evaluate THC recovery from compressible cellulose collecting element material pre-treated with Tween-20 at a concentration of 1.0% Tween-20 treated cellulose collecting elements in which the saliva was centrifuged off the collecting elements into the preservative solution compared to soaking the collecting elements in the preservative solution for one hour before centrifugation. This centrifugation of the saliva into the preservative solution more closely resembles collection in which the saliva is squeezed off of the collector into the preservative solution, instead of the collector being stored in the preservative solution.

The experiment was carried out as in the previous example except the additional condition of centrifuging of the saliva off the collecting element into the 800 uL of preservative solution immediately after applying the saliva to the collecting element. For this condition, we corrected for the saliva that could not be spun off the collecting element by weighing the collecting element after the addition of the 400 uL of saliva and after the centrifugation step. We applied this correction factor to the LC/MS/MS concentration determined from the saliva spun into the preservative buffer samples only. We do not apply this correction factor to the collecting elements soaked in the preservative buffer for one hour since it is assumed that the saliva comes into equilibrium with the preservative buffer prior to the centrifugation step.

### EXAMPLE 7

### Results

| | | | | Saliva | | |
|---|---|---|---|---|---|---|
| | | | % | volume | Corrected | % |
| Sample | Value | Mean | Recovery | recovered | concentration | Recovery |
| Saliva-1 | 5.09 | 5.0 | 100 | | | |
| Saliva-2 | 4.93 | | | | | |
| Saliva-3 | 4.89 | | | | | |
| Saliva-4 | 4.81 | | | | | |
| Saliva-5 | 5.27 | | | | | |
| 1% Tween cellulose | 4.16 | 4.25 | 85 | | | |
| soaked in preservative-1 | | | | | | |
| 1% Tween cellulose | 4.08 | | | | | |
| soaked in preservative-2 | | | | | | |
| 1% Tween cellulose | 4.54 | | | | | |
| soaked in preservative-3 | | | | | | |
| 1% Tween cellulose | 4.53 | | | | | |
| soaked in preservative-4 | | | | | | |
| 1% Tween cellulose | 3.92 | | | | | |
| soaked in preservative-5 | | | | | | |
| 1% Tween cellulose | 3.05 | 3.14 | | 300 uL | 4.1 | 83 |
| spun into preservative-1 | | | | | | |
| 1% Tween cellulose | 3.4 | | | | | |
| spun into preservative-2 | | | | | | |
| 1% Tween cellulose | 3.04 | | | | | |
| spun into preservative-3 | | | | | | |
| 1% Tween cellulose | 3.2 | | | | | |
| spun into preservative-4 | | | | | | |
| 1% Tween cellulose | 3.03 | | | | | |
| spun into preservative-5 | | | | | | |

After applying the correction factor to the saliva samples that were spun into preservative, there was no significant difference between the THC recovery for either method.

### EXAMPLE 8

### Comparison of Various Detergents on THC Recovery

The following tests evaluate the recovery of THC from compressible type cellulose collecting elements that were pre-treated with various cationic, anionic, zwitterionic and nonionic detergents.

The experiment was carried out as in the previous examples with the following exceptions:
1) only the Ultracell cellulose collecting elements were pretreated with the various detergents
2) replicates of 4 were run for each detergent condition
3) the concentration of THC in saliva was spiked at 48 ng/mL
4) instead of soaking the collecting element in the preservative solution for 60 minutes prior to the centrifugation, the collecting elements were spun down immediately into the preservative solution after adding the saliva to the collecting element materials. A correction factor was then applied to correct for the remaining saliva that could not be centrifuged from the collecting element.

The seven detergents tested are:
1) N -Dodecyl-N,N-dimethyl-3-ammonio-1-propanesulfonate (SB-12) Sigma, D4516-5G
2) Deoxycholic acid sodium salt Sigma, D6750
3) Pluronic F-127, Sigma P2443
4) Brij 58, Sigma, P5884

5) Lauryl sulfate (SDS), Sigma, L-4509
6) 3-[(3-Cholamidopropyl)dimethylammonio]propanesulfonic acid (CHAPS), Pierce, 28300
7) Cetyltrimethylammonium Bromide (CTAB), CalBiochem, 219375

### EXAMPLE 8

### Results

| | | Calculated | | Mean |
|---|---|---|---|---|
| Detergent | | Concentration | | Corrected % |
| Type | Sample Name | (ng/mL) | Mean | Recovery |
| Zwitterionic | SB-12 treated cellulose pad-1 | 5.65 | 6.1 | 45 |
| | SB-12 treated cellulose pad-2 | 6.2 | | |
| | SB-12 treated cellulose pad-3 | 6.05 | | |
| | SB-12 treated cellulose pad-4 | 6.45 | | |
| Non-ionic | Brij 58 treated cellulose pad-1 | 12.8 | 10.8 | 80 |
| | Brij 58 treated cellulose pad-2 | 9.44 | | |
| | Brij 58 treated cellulose pad-3 | 10.9 | | |
| | Brij 58 treated cellulose pad-4 | 9.88 | | |
| Zwitterionic | CHAPS treated cellulose pad-1 | 3.61 | 3.6 | 26 |
| | CHAPS treated cellulose pad-2 | 2.86 | | |
| | CHAPS treated cellulose pad-3 | 3.77 | | |
| | CHAPS treated cellulose pad-4 | 4.04 | | |
| Anionic | Deoxycholate treated cellulose pad-1 | 5.43 | 5.9 | 44 |
| | Deoxycholate treated cellulose pad-2 | 5.25 | | |
| | Deoxycholate treated cellulose pad-3 | 6.79 | | |
| | Deoxycholate treated cellulose pad-4 | 5.99 | | |
| Non-ionic | F-127 treated cellulose pad-1 | 3.86 | 4.0 | 30 |
| | F-127 treated cellulose pad-2 | 4.52 | | |
| | F-127 treated cellulose pad-3 | 3.52 | | |
| | F-127 treated cellulose pad-4 | 4.06 | | |
| Cationic | CTAB treated cellulose pad-1 | 3.92 | 4.7 | 35 |
| | CTAB treated cellulose pad-2 | 5.22 | | |
| | CTAB treated cellulose pad-3 | 4.91 | | |
| | CTAB treated cellulose pad-4 | 4.87 | | |
| Anionic | SDS treated cellulose pad-1 | 6.77 | 6.6 | 49 |
| | SDS treated cellulose pad-2 | 6.29 | | |
| | SDS treated cellulose pad-3 | 6.61 | | |
| | SDS treated cellulose pad-4 | 6.75 | | |
| | Saliva-1 | 18 | 18.0 | 100 |
| | Saliva-2 | 17 | | |
| | Saliva-3 | 18.9 | | |

The above experiment yielded 80% recovery of THC with pre-treatment of the cellulose collecting elements with Brij 58 because it is a non-ionic detergent like Tween-20. All other detergent pre-treatments yielded lower percent recoveries. Thus we identified another detergent which performs similarly to Tween-20.

As described with reference to the embodiment of FIGS. 7A and 7B, an indicator wick 713 may be employed to indicate to the user when a sufficient amount of sample fluid has been absorbed by the collecting element 710, i.e., the collecting element. In particular, the indicator wick 713 may contact the collecting element 710 so that the indicator wick 713 also receives sample fluid when the collecting element 710 has become saturated, or absorbed a sufficient amount of sample fluid. The sample fluid then causes the indicator wick 713 to exhibit a physical change, such as a color change, which signals the user. The following examples further illustrate aspects of embodiments of an indicator wick.

### EXAMPLE 9

### Indicator Wick Study with Untreated Collecting Elements Tested on Human Volunteers

The following tests evaluate the length of time and the minimum volume at which indicator wicks change color when untreated collecting elements are tested with human volunteers. Polyolefin fiber collecting elements used in this experiment are cylinder shaped with dimensions of OD-12.22 mm, length of 16.14 mm, and a capacity of approximately 1.8 mL (from Filtrona Fibertec, Va.). Fiber wicks used in this experiment are cylinder shaped with dimensions of OD-2.05 mm and cut to a length of 15 mm. The wicks were marked at 10 mm with marker ink that bleeds when wet. The wicks were placed into the polypropylene sticks and the polyolefin fiber collecting elements were glued onto the polypropylene sticks containing the indicator wicks. Human volunteers were instructed to collect oral fluid via one of the following methods: swab each cheek for 30 seconds then hold the collecting element in the cheek until the indicator wick changed color (Collection type “A”), and swab 3 times around both cheeks then hold under the tongue for 2 minutes and repeat process until the indicator wick changed color (Collection type “B”). Timing of collection began when volunteer began swabbing and ended when the indicator wick changed color. Collectors were weighed before and after oral fluid collection. The difference in the weights determined the volume of oral fluid collected.

### EXAMPLE 9

### Results

| | | Oral fluid | |
|---|---|---|---|
| | | Volume | Time of |
| | | Collected | collection |
| | Sample # | (mL) | (min) |
| Collection Type “A” | 1 | 0.6 | 7.5 |
| | 2 | 0.8 | 2.0 |
| | 3 | 0.8 | 4.5 |
| | 4 | 1 | 3.0 |
| | 5 | 1.2 | 6.0 |
| | 6 | 1.2 | 2.0 |
| Collection Type “B” | 7 | 1 | 6.0 |
| | 8 | 1 | 7.0 |
| | 9 | 1.1 | 2.0 |
| | 10 | 1.1 | 10.8 |
| | 11 | 1.4 | 5.0 |
| | 12 | 1.5 | 9.0 |
| | 13 | 1.3 | 2 |
| | 14 | 1.3 | 3 |
| | 15 | 1.1 | 9 |
| | 16 | 1.3 | 4 |
| | 17 | 0.8 | 1 |
| | 18 | 1.1 | 2.5 |
| | 19 | 1.1 | 4.5 |
| | 20 | 1.1 | 5 |

The minimum amount of oral fluid collected with the Collection Type A was 0.6 mLs (30% capacity of collecting element) while the minimum amount of oral fluid collected with the Collection Type B was 0.8 mLs (53% capacity of collecting element). The shortest period of time for the wick to change color was 2 minutes for Collection Type A and 1 minute for Collection Type B.

### EXAMPLE 10

### Indicator Wick Study with Salt and PEG 400 Treated Collecting Elements Tested on Human Volunteers

The following tests evaluate the length of time and the minimum volume at which indicator wicks change color when salt and PEG 400 treated collecting elements are tested with human subjects. Polyolefin fiber collecting elements used in this experiment are cylinder shaped with dimensions of OD-12.22 mm, length of 16.14 mm, and a capacity of approximately 1.8 mL (from Filtrona Fibertec, Va.). Fiber wicks used in this experiment are cylinder shaped with dimensions of OD-2.05 mm and cut to a length of 15 mm. The polyolefin fiber collecting elements were pretreated with a solution containing the following: 0.375% PEG 400, 2.5% sodium chloride, 0.0715% potassium sorbate, 0.0715% sodium benzoate, 0.2145% citric acid, and 0.12% sodium hydroxide. The polyolefin fiber collecting elements were pretreated by saturating the collecting elements with 2 mL (111% of collecting element capacity) of the salt and PEG 400 solution and by allowing them to dry overnight at 37° C. The wicks were marked at 10 mm with marker ink that bleeds when wet. The wicks were placed into the polypropylene sticks and the polyolefin fiber collecting elements were glued onto the polypropylene sticks containing the indicator wicks. Human volunteers were instructed to collect oral fluid via one of the following methods: swab each cheek for 30 seconds then hold the collecting element in the cheek until the indicator wick changed color (Collection type “A”), swab 3 times around both cheeks then hold under the tongue for 2 minutes and repeat process until the indicator wick changed color (Collection type “B”), or to hold the collecting element under the tongue until the wick changed color (Collection type “C”). Timing of collection began when volunteer began swabbing and ended when the indicator wick changed color. Collection sticks were weighed before and after oral fluid collection. The difference in the weights determined the volume of oral fluid collected.

### EXAMPLE 10

### Results

| | Sample | Oral fluid Volume | Time of Collection |
|---|---|---|---|
| | # | Collected (mL) | (min) |
| Collection Type “A” | 1 | 1.2 | 0.5 |
| | 2 | 1.0 | 1.0 |
| | 3 | 1.0 | 0.5 |
| | 4 | 0.9 | 0.5 |
| Collection Type “B” | 5 | 0.4 | 0.5 |
| | 6 | 0.7 | 0.5 |
| | 7 | 0.6 | 0.5 |
| | 8 | 1.1 | 1.0 |
| Collection Type “C” | 9 | 0.6 | 1.0 |
| | 10 | 1.1 | 0.5 |

The minimum volume of oral fluid collected in the above experiment ranged from 0.4 mL to 0.9 mL in 1 minute or less. Collection type “A” produced a minimum collection volume of 0.9 mL (60% collecting element capacity). Collection type “B” produced a minimum collection volume of 0.4 mL (27% collecting element capacity) Collection type “C” produced a minimum collection volume of 0.6 mL (40% collecting element capacity).

It will be apparent to those skilled in the art that, while the present invention has been disclosed with reference to certain embodiments, numerous modifications, alterations and changes to the described embodiments are possible without departing from the spirit or scope of the present invention, as defined in the appended claims. For example, in alternative embodiments, a sufficient volume of sample fluid required for testing may be collected with more than one collecting element. Accordingly, it is intended that the present invention not be limited to the described embodiments, but that it has the full scope defined by the language of the following claims, and equivalents thereof.

What is claimed is:

1. A system for collecting a sample of bodily fluid, the system comprising:
a collector having a sample collecting element, the sample collecting element being adapted to receive the sample when the collector is in a first configuration and to release the sample when the collector transitions from the first configuration to a second configuration;
a container adapted to receive and store the sample released from the collector; and
an extractor operably connected to the container, the extractor receiving the collector and having a passage providing fluid communication between the collector and the container,
wherein the collector, when received by the extractor, is operable to transition from the first configuration to the second configuration and release a volume of the sample.

2. The system according to claim 1, wherein the extractor comprises an extractor cavity for receiving at least a part of the collector holding the sample.

3. The system according to claim 2, wherein the collector comprises an absorbent material, the absorbent material being expandable when the collector is in the first configuration and compressed when the collector is in the second configuration.

4. The system according to claim 3, wherein the collector further comprises a plunger, the absorbent material being positioned on an end of the plunger, and the plunger being operable to compress the absorbent material and transition the collector from the first configuration to the second configuration.

5. The system according to claim 4, further comprising an extracting structure, wherein the plunger is movable relative to the extracting structure to compress the absorbent material against the extracting structure.

6. The system according to claim 4, wherein the collector further comprises an end section and a stem extending from the end section, the plunger having an interior cavity for receiving the stem and being guidable over the stem to compress the absorbent material against the end section.

7. The system according to claim 6, wherein the collector further comprises an overflow chamber positioned in the stem, the overflow chamber having an overflow opening to receive an excess volume of the sample when the collector is operated to transition from the first configuration to the second configuration.

8. The system according to claim 7, further comprising a valve that determines a volume of fluid received by the overflow chamber.

9. The system according to claim 8, wherein the overflow chamber extends from the overflow opening positioned at the end section to a valve opening at an opposing end of the stem, and the valve comprises a stopper that is positioned in the interior cavity of the plunger and blocks the valve opening to substantially prevent a flow of fluid through the overflow opening when the collector is in the second configuration.

10. The system according to claim 2, wherein the extractor further comprises a cap that removably covers the extractor cavity.

11. The system according to claim 10, wherein the cap engages the extractor with a screw-thread, and the cap is operable to release the sample from the collector as the cap is screwed onto the extractor cavity.

12. The system according to claim 1, wherein the collector further comprises a handle, at least a portion of the handle being detachable.

13. The system according to claim 1, further comprising an overflow chamber positioned in at least one of the collector and the extractor, the overflow chamber having an overflow opening to receive excess sample when the collector is operated to transition from the first configuration to the second configuration.

14. The system according to claim 13, further comprising a valve that determines a volume of fluid received by the overflow chamber.

15. The system according to claim 1, wherein the extractor forms an interior component of the container.

16. The system according to claim 1, wherein the extractor is detachably connected to the collector and the container.

17. The system according to claim 1, further comprising at least one screen in the passage between the collector and the container.

18. The system according to claim 1, further comprising a breakable seal between the extractor and the container, wherein a receiving chamber for receiving the sample from the collector is formed in the extractor between the seal and the collector, and the extractor has a breaking component for breaking the seal and releasing the sample from the receiving chamber into the container.

19. The system according to claim 18, further comprising an overflow chamber positioned in at least one of the collector and the extractor, the overflow chamber having an overflow opening to receive an excess volume of the sample when the collector is operated to transition from the first configuration to the second configuration.

20. The system according to claim 19, further comprising a valve operable to close the overflow opening before the sample is released from the receiving chamber into the container.

21. The system according to claim 20, wherein the valve closes the overflow opening when the collector is rotated relative to the extractor for a specified number of degrees.

22. The system according to claim 1, wherein the container has an indicator indicating a volume of sample released from the collector into the container.

23. The system according to claim 22, wherein the indicator is a window showing a level of fluid in the container.

24. The system according to claim 1, wherein the collector is operable to occupy states between the first and second configurations, and a part of the volume of the sample is released.

25. The system according to claim 1, wherein the container further contains a diluent.

26. The system according to claim 1, further comprising an indicator that indicates when the sample collecting element collects a sufficient volume of sample fluid.

27. The system according to claim 26, wherein the indicator changes color when the sample collecting element collects a sufficient volume of sample fluid.

28. The system according to claim 26, wherein the indicator is a wick in contact with the sample collecting element, the wick exhibiting a physical change when contacting the sample received by the sample collecting element.

29. A method for collecting a sample of bodily fluid, the method comprising:
receiving, with a collector, a sample of bodily fluid, the collector having a first configuration and a second configuration and being adapted to hold the sample when the collector is in the first configuration and to release the sample when the collector transitions from the first configuration to the second configuration;
connecting the collector operably to an extractor, the extractor having a passage providing fluid communication between the collector and a container, and the container being adapted to receive and store the sample released from the collector; and
operating the collector to transition the collector from the first configuration to the second configuration to release a volume of the sample.

30. The method according to claim 29, wherein the step of operating the collector to release the sample comprises compressing an absorbent material of the collector, the absorbent material being expandable when the collector is in the first configuration and compressed when the collector is in the second configuration.

31. The method according to claim 30, wherein the step of operating the collector comprises compressing the absorbent material with a plunger, the plunger being movable to compress the absorbent material and to transition the collector from the first configuration to the second configuration.

32. The method according to claim 31, wherein the step of compressing the absorbent material with a plunger comprises compressing the absorbent material between the plunger and an end section by guiding the plunger over a stem, the end section being proximal to the extractor and a stem extending from the end section away from the extractor, and the plunger having an interior cavity for receiving the stem.

33. The method according to claim 32, wherein the collector further comprises an overflow chamber positioned in the stem, the overflow chamber having an overflow opening to receive an excess volume of the sample when the collector is operated to transition from the first configuration to the second configuration.

34. The method according to claim 33, further comprising a valve that determines a volume of fluid received by the overflow chamber.

35. The method according to claim 34, wherein the overflow chamber extends from the overflow opening positioned at the end section to a valve opening at an opposing end of the stem, and the valve comprises a stopper that is positioned in the interior cavity of the plunger and blocks the valve opening to substantially prevent a flow of fluid through the overflow opening when the collector is in the second configuration.

36. The method according to claim 29, wherein the step of connecting the collector operably to an extractor comprises receiving, into an extractor cavity, at least a part of the collector holding the sample.

37. The method according to claim 36, wherein the step of operating the collector further comprises covering the extractor cavity with a removable cap and compressing the absorbent material received into the extractor cavity by operation of the cap.

38. The method according to claim 34, wherein the step of operating the collector further comprises covering the extractor cavity with the cap via a screw-thread and compressing the absorbent material received into the extractor cavity by screwing the cap over the extractor cavity.

39. The method according to claim 30, wherein the collector further comprises a handle extending from the absorbent material.

40. The method according to claim 39, further comprising detaching the at least a portion of the handle.

41. The method according to claim 29, further comprising an overflow chamber positioned in at least one of the collector and the extractor, the overflow chamber having an overflow opening to receive excess sample when the collector is operated to transition from the first configuration to the second configuration.

42. The method according to claim 41, further comprising a valve that determines a volume of fluid received by the overflow chamber.

43. The method according to claim 29, wherein the step of connecting the collector operably to an extractor comprises detachably connecting the extractor to the collector and the container.

44. The method according to claim 29, wherein the extractor forms an interior component of the container.

45. The method according to claim 29, further comprising at least one screen in the passage between the collector and the container.

46. The method according to claim 29, further comprising breaking a seal between the extractor and the container, wherein a receiving chamber for receiving the sample from the collector is formed in the extractor between the seal and the collector, and the extractor has a breaking component for breaking the seal and releasing the sample from the receiving chamber into the container.

47. The method according to claim 46, further comprising an overflow chamber positioned in at least one of the collector and the extractor, the overflow chamber having an overflow opening to receive an excess volume of the sample when the collector is operated to transition from the first configuration to the second configuration.

48. The method according to claim 47, further comprising closing a valve to close the overflow opening before the sample is released from the receiving chamber into the container.

49. The method according to claim 48, wherein the step of closing a valve comprises rotating the extractor for a specified number of degrees to align the valve with a valve closure element.

50. The method according to claim 26, further comprising determining, with an indicator, a volume of sample released from the collector into the container.

51. The method according to claim 47, wherein the indicator is a window showing a level of fluid in the container.

52. The method according to claim 29, wherein the step of operating the collector comprises operating the collector to occupy states between the first and second configurations to release a part of the volume of the sample.

53. The method according to claim 29, further comprising providing a diluent in the container.

54. A method of collecting an oral fluid specimen from an oral cavity for testing comprising the steps of:
(a) inserting a compressible, detergent-treated collecting element into the oral cavity;
(b) contacting the collecting element with an oral fluid within the oral cavity for a sufficient time to collect an oral fluid sample and without masticating the collecting element; and
(c) removing the collecting element from the oral cavity.

55. A method of claim 54, comprising, after removing the collecting element from the oral cavity, the step of preserving the collecting element for subsequent recovery of the collected specimen from the collecting element for testing.

56. A method of claim 55, wherein the step of preserving the collecting element comprises storing the collecting element in a preservative solution after the collecting element is removed from the oral cavity.

57. A method of claim 54 or 55, further comprising the step of compressing the collecting element to recover the collected specimen from the collecting element.
